(19) United States
(12) Patent Application Publication
(10) Pub. No.: US 2020/0190507 A1
(43) Pub. Date: Jun. 18, 2020

(54) Encoded Solid Phase Compound Library with Polynucleotide Based Barcoding

(71) Applicant: The Scripps Research Institute, La Jolla, CA (US)

(72) Inventors: Brian M. Paegel, West Palm Beach, FL (US); Andrew B. MacConnell, Jupiter, FL (US); Thomas Kodadek, Jupiter, FL (US); Patrick J. McEnaney, North Palm Beach, FL (US)

(21) Appl. No.: 16/349,097

(22) PCT Filed: Nov. 9, 2017
(86) PCT No.: PCT/US17/60870

§ 371 Date: May 10, 2019

Related U.S. Application Data
(60) Provisional application No. 62/420,303, filed on Nov. 10, 2016.

(51) Int. Cl. C12N 15/10

(52) U.S. Cl. CPC C12N 15/1065

(57) ABSTRACT

Provided herein are polynucleotide encoded chemical libraries comprising one or more bead members, wherein the beads comprise: a chemical moiety comprising a compound library member; a polynucleotide moiety comprising an oligonucleotide encoding the compound library member, and a barcode identifying the bead; and a linking moiety, linking the chemical moiety to the polynucleotide moiety. Also provided herein are methods of making and using the polynucleotide barcoded chemical libraries, as well as kits comprising the barcoded chemical library.

### CROSS-REFERENCE TO RELATED APPLICATIONS

The subject patent application claims the benefit of priority to U.S. Provisional Patent Application No. 62/420,303 (filed Nov. 10, 2016). The full disclosure of the priority application is incorporated herein by reference in its entirety and for all purposes.

### GOVERNMENT RIGHTS

This invention was made with government support under DP2OD008535 awarded by United States National Institute of Health (NIH), and N66001-14-2-4057 awarded by United States Department of Defense DARPA. The government has certain rights in the invention.

### FIELD OF THE INVENTION

The present disclosure relates to screening and production of compounds, including drug development.

### BACKGROUND OF THE DISCLOSURE

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Drug discovery remains a costly and specialized pursuit limited to a few major research facilities. At the heart of the problem is the compound library, a collection of molecular entities each inhabiting a single microtiter plate well and ranging in size from several thousand to several million different species. The management of these collections comes at enormous cost in terms of automation, analysis, and manpower, as does generation of molecular diversity by way of serial synthesis. These constraints constitute key technological barriers to transforming high throughput screening (HTS) based small molecule discovery into a distributable and thereby economical enterprise.

Thus there remains a need in the art for new devices and methods for screening compounds cost-effectively, efficiently, and with high accuracy,

### SUMMARY OF THE DISCLOSURE

Various embodiments disclosed herein include a polynucleotide encoded chemical library comprising one or more bead members, wherein the beads comprise: a chemical moiety comprising a compound library member; a polynucleotide moiety comprising: an oligonucleotide whose sequence encodes the compound library member, and a barcode identifying the bead; and a linking moiety, linking the chemical moiety to the polynucleotide moiety. In one embodiment, the barcode identifying the bead is an oligonucleotide. In one embodiment, the polynucleotide and/or oligonucleotide are composed of DNA nucleotides. In one embodiment, the polynucleotide encoded chemical library comprises two or more bead members having the identical compound library member, identical oligonucleotide sequences encoding the compound library member, but different barcodes identifying each bead. In one embodiment, the presence of identical compound library members on more than one bead while having different barcodes identifying each bead enables discriminating between the two or more beads carrying the same compound library member. In one embodiment, the barcode identifying the bead comprises an oligonucleotide having a length of 2 to 20 nucleotides. In another embodiment, the barcode identifying the bead comprises an oligonucleotide having a length of 2 to 50 nucleotides. In one embodiment, the polynucleotide moiety is synthesized in solid phase on the beads. In one embodiment, the oligonucleotide encoding the compound library member is ligated in parallel with the compound library member synthesis. In one embodiment, bead barcoding can occur at any point during the synthesis. In one preferred embodiment, bead barcoding occurs “up front” before the encoded synthesis. In another embodiment, bead barcoding occurs after encoded synthesis. In yet another embodiment, bead barcoding occurs discontinuously, wherein portions of the barcode are installed before and after the synthesis.

In one embodiment, polynucleotide encoded split-and-pool synthesis proceeds with alternating steps of monomer coupling followed by oligonucleotide ligation-based encoding. In one embodiment, the oligonucleotide sequences encoding the compound library member and/or identifying the bead are thermodynamically optimized. In one embodiment, the oligonucleotide sequences encoding the compound library member and/or identifying the bead possess Hamming string distances ≥3. In one embodiment, the oligonucleotide sequences encoding the compound library member and/or identifying the bead has a total read length <100 bases for facile sequencing. In one embodiment, the oligonucleotide sequences encoding the compound library member and/or identifying the bead are thermodynamically optimized. In one embodiment, the linker comprises a chromophore. In one embodiment, the chromophore is coumarin. In one embodiment, the linker comprises a chemical moiety that enhances mass spectrometric ionization efficiency. In one embodiment, the chemical moiety is arginine. In one embodiment, the linker comprises an alkyne for copper catalyzed azide-alkyne cycloaddition click chemistry. In one embodiment, the barcode identifying the bead enables removal of false positive hits. In one embodiment, the polynucleotide sequencing data obtained after a screen reveals both the structure of the hit compounds and provide hit reproducibility data that rejects false positives. In one embodiment, the rejection of false positives justifies further downstream re-synthesis and functional characterization. In one embodiment, the bead count correlates with molecular properties such as potency and/or selectivity. In one embodiment, the bead displays compound library member, barcode region, and compound library member structure-encoding region as shown in FIG. 1. In one embodiment, the bead displays compound library member, barcode region, and structure-encoding region as shown in FIG. 4.

Various embodiments disclosed herein also include methods of combinatorial screening comprising the steps of: (i) incubating a fluorescently labeled protein with a polynucleotide-encoded chemical library comprising a plurality of encoded compound bead members, wherein the beads comprise a chemical moiety comprising a compound library member, a polynucleotide moiety comprising an oligonucleotide encoding the compound library member structure, and a barcode identifying the bead, and a linking moiety, variously linking bead, compound library member, and encoding polynucleotide; (ii) washing the beads to remove excess unbound protein; (iii) sorting and detecting the beads that have bound to the labeled protein; (iv) amplifying the compound library member structure-encoding polynucleotide sequences of the hit beads using PCR; (v) sequencing the polynucleotide moiety; and (vi) decoding the hit compound library member structures based on the sequence of the structure-encoding oligonucleotide. In one embodiment, the barcode identifying the bead is an oligonucleotide. In one embodiment, the polynucleotide and/or oligonucleotide is a DNA oligonucleotide. In one embodiment, the target binding during screening is deemed to be authentic if multiple beads containing the same compound library member are identified as hits and/or more than one bead-specific barcode identifies the same compound library member as a hit.

Various embodiments disclosed herein further include kits for combinatorial screening comprising: a polynucleotide encoded chemical library comprising one or more bead members, wherein the beads comprise a chemical moiety comprising a compound library member, a polynucleotide moiety comprising an oligonucleotide encoding the compound library member structure, and a barcode identifying the bead and a linking moiety, variously linking bead, compound library member, and encoding polynucleotide; and instruction for using the kit for combinatorial screening. In one embodiment, the instruction for using the kit is a printed instruction, video instruction, and/or audio instruction.

Other embodiments disclosed herein include methods of yielding a panel of molecular diagnostics for detecting the presence of a disease state comprising: (i) providing a sample from a patient afflicted with the disease, and sample from a control individual not afflicted the disease; (ii) screening the samples against a polynucleotide encoded chemical library; (iii) utilizing a fluorescent tag to label hit compound beads for fluorescence-activated cell sorting (FACS); (iv) PCR amplification of the polynucleotides encoding the structures of the hit compound library members and subsequent deep sequencing to determine the structure of the hit compounds and each hit's occurrence frequency; (v) separating the disease-afflicted patient hits from the control, unafflicted patient hits; and (vi) resynthesizing the disease-afflicted patient hits to yield a diagnostic panel for the disease. In one embodiment, the disease is active tuberculosis (ATB). In one embodiment, the control individual is someone who has noninfectious/latent TB (LTB). In one embodiment, the sample is a serum sample. In one embodiment, the fluorescent tag is anti-human IgG. In one embodiment, the diagnostic panel of drug molecules comprises thermally stable and economically produced small molecules. In one embodiment, the patient samples are pools of patients presenting as the same disease or control state.

Other embodiments disclosed herein include a device, comprising a chemical moiety linked to a polynucleotide moiety, wherein the polynucleotide moiety comprises a barcode region and a binding region. In one embodiment, the binding region binds with specificity to a compound library member. In one embodiment, the barcode region indicates a specific bead. In one embodiment, the device is a screening device.

Other features and advantages of the invention will become apparent from the following detailed description, taken in conjunction with the accompanying drawings, which illustrate, by way of example, various embodiments of the invention.

### DESCRIPTION OF THE DRAWINGS

Exemplary embodiments are illustrated in referenced figures. It is intended that the embodiments and figures disclosed herein are to be considered illustrative rather than restrictive.

FIG. 1 depicts, in accordance with embodiments herein, split-and-pool ligation strategy for DNA-based bead specific barcoding. DNA-encoded synthesis entails coupling enzymatic synthesis of an encoding oligonucleotide with corresponding monomer coupling steps on a bifunctional resin that supports parallel synthesis of both species. The encoding region corresponds with the compound library member structural elements. The tag is bounded by primer binding sequences. In addition to chemistry-encoding elements, one can employ the split-and-pool strategy with ligation reactions to generate a bead-specific barcode region (here shown before the encoding region). With four different sequences shown on the left and four different sequences shown on the right, 16 different barcodes are possible for the purposes of distinguishing beads displaying identical compounds, which would otherwise be indistinguishable due to the compound encoding regions being identical.

FIG. 2 depicts, in accordance with embodiments herein, FACS-based high-throughput library screening workflow. The encoded library is treated with Starting Block to block sites of non-specific protein adsorption, then incubated with the Alexa Fluor 647-labeled streptavidin (SA647) target and washed. The labeled beads are sorted by FACS. The hit beads are collected as a batch, DNA encoding tag sequences are amplified in PCR and sequenced using the Ion Torrent/Ion Proton platform to yield a table of sequences (depicted as the 4-digit identifiers).

FIG. 3 depicts, in accordance with embodiments herein, affinity measurement of compound 2 for streptavidin. Fluorescein-labeled 2 (10 nM) was incubated at varying concentrations of streptavidin and the resulting fluorescence anisotropy determined. The dissociation constant for the compound 2—streptavidin complex was determined to be ˜12 μM. Similar binding measurements of 2 with choleratoxin B subunit (CTOX) or proteasome subunit Rpn13 yielded no detectable binding.

FIG. 4 depicts, in accordance with embodiments herein, DNA-encoded solid-phase synthesis and bead-specific barcoding. (a) The DNA-encoded solid-phase synthesis bifunctional resin linker displays amine sites for compound synthesis and DNA headpiece sites (HDNA, a tether that covalently joins the two DNA strands) for enzymatic ligation of encoding oligonucleotides. The encoding tag contains a synthesis-encoding region and bead barcoding region flanked by forward and reverse primer binding modules. After ligation of the forward primer sequence, each monomer coupling step accompanies an enzymatic cohesive end ligation that installs a dsDNA encoding module. A submonomer approach includes various main chain scaffold structures and amine side chains. Corresponding encoding modules appear in the same color. After encoded synthesis, combinatorial ligation of two additional encoding modules assigns a bead-specific barcode, and reverse primer ligation completes the encoding tag. (b) Bead-specific barcodes distinguish beads that harbor identical compounds, which would otherwise display identical DNA sequences. (c) Combinatorial ligation of i sequence modules in the first bead-specific barcoding position (cyan hues) and j sequence modules in the second position (green hues) yields i×j possible unique bead-specific barcodes.

FIG. 5 depicts, in accordance with embodiments herein, hit compound validation and native antigen identification. (a) Beads displaying compound 2-B bound statistically significantly more ATB discovery serum pool lgG compared to the NCL discovery serum pool IgG over a wide range of [serum]. Competition binding analysis of 2-B revealed competitive binding of hypervirulent culture filtrate proteins (CFP, 250 μg/mL) derived from several hypervirulent Mtb strains (HN878, CDC1551. H37Rv), while E. coli and Mtb lysates weakly competed (b). Purified Mtb proteins Ag85A and Ag85B competed (the latter strongly so) though the recombinantly expressed forms were unreactive. (c) Competition titration analysis of native Ag85A and Ag85B with beads displaying 2-B revealed selective reactivity with Ag85B. (d) ELISA analysis of all serum samples using non-specifically immobilized native Ag85B as the antigen yielded 22% diagnostic sensitivity and 100% specificity.

### DETAILED DESCRIPTION

All references, publications, and patents cited herein are incorporated by reference in their entirety as though they are fully set forth. Unless defined otherwise, technical and scientific terms used herein have the same meaning as commonly understood by one of ordinary skill in the art to which this invention belongs. Hornyak, et al., Introduction to Nanoscience and Nanotechnology, CRC Press (2008); Singleton et al., Dictionary of Microbiology and Molecular Biology 3rd ed., J. Wiley & Sons (New York, N.Y. 2001); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4th ed., Cold Spring Harbor Laboratory Press (Cold Spring Harbor, N.Y. 2012), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

The terms “polynucleotide” and “oligonucleotide,” used interchangeably herein, refer generally to linear polymers of natural or modified nucleosides, including deoxyribonucleosides, ribonucleosides, alpha-anomeric forms thereof, and the like, usually linked by phosphodiester bonds or analogs thereof ranging in size from a few monomeric units, e.g. 2-4, to several hundreds of monomeric units. When a polynucleotide is represented by a sequence of letters, such as “ATGCCTG,” it will be understood that the nucleotides are in 5′->3′ order from left to right. Polynucleotide as used herein also includes a basic sugar-phosphate or sugar-phosphorothioate polymers.

In accordance with various embodiments herein, the term “DNA”, or deoxyribonucleic acid, are used variously in conjunction with embodiments and terms described herein such as “DNA-encoded libraries,” or “DNA moiety,” or “DNA barcode,” for example. As readily apparent to one of skill in the art, various other compounds and structures, such as polynucleotides, or RNA, for example, may also be used in conjunction with various embodiments described herein, and the invention is in no way only limited to DNA.

As used herein, the term “compound library” refers to a collection of two or more compounds. In one embodiment, the compound is a small organic or inorganic molecule. In another embodiment, the compound can be a peptide, oligomer, or polymer. As used herein, the term “compound library member” refers to a member of the compound library.

As disclosed herein, a method was developed to encode solid-phase synthesis using enzymatic ligation of DNA oligonucleotides. See MacConnell et al, ACS Combinatorial Science, 2015, 17, 518-534, which is incorporated herein by reference in its entirety. In brief, large DNA-encoded bead libraries were generated by split-and-pool synthesis. Each split comprises monomer coupling followed by enzymatic ligation to encode the monomer coupled. Each bead of the resulting split-and-pool library displayed many copies of a compound and a PCR-amplifiable DNA tag that described the compound structure. Such libraries could then be used for conventional bead-based screening for ligands as well as droplet-based functional screening in emulsions or microfluidic devices. One problem with this technology, as well as other currently available bead screening technologies, is that the false positive rate is high. It is difficult to distinguish the sequences representing true hits from the much higher number of sequences that encode false positives. In other words, the noise is overwhelming. The inventors saw a need in the art to solve this problem.

As described herein, in accordance with the various embodiments herein, the inventors have developed a novel technology that encodes not only the compound structure on the bead, but also assigns a barcode to the bead itself. Presently available DNA-encoded libraries are synthesized in solution and screened in solution as well. In contrast, the bead-specific barcode DNA-encoded libraries disclosed herein are created on beads and screened on beads. Bead screening involves incubating a labeled protein with a large number of beads, then detecting beads that have picked up the label (usually a fluorescent tag). The notion is that these beads display a compound that is a good ligand for the protein target. However, the false positive rate in bead screening is quite high. In accordance with various embodiments herein, when beads are assigned a barcode, and when redundant libraries (i.e., several different beads display the same compound) are used, the hits that are found on more than one bead are always bona fide ligands. Thus, in one embodiment, the present disclosure provides a bead screening technique that allows a way of determining if the same compound was identified as a hit on more than one bead.

In one embodiment, the present invention provides DNA barcoding technology, wherein the DNA barcoding adds a bead-specific tag to each bead that is read out in the deep sequencing experiment. Thus, the present disclosure concerns the use of serial oligonucleotide ligation not only to encode the compound structure on the bead, but also to assign a barcode to the bead itself. At any point in the library synthesis, split-and-pool methods may be applied to ligation steps only in order to generate these bead-specific DNA barcodes such that two beads may display identical compound and thereby display the same DNA sequence describing the identical compound, however the bead-specific barcode enables discrimination between the two beads. The number of different barcodes possible is dictated by the number of individual elements (in this case the number of different sequences) raised to the power of the number of pooling steps.

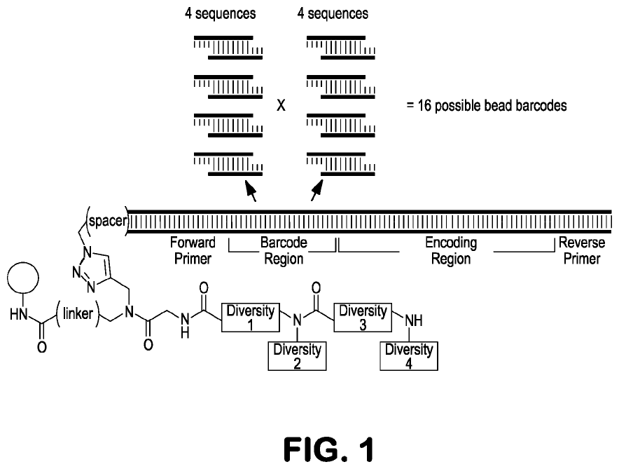

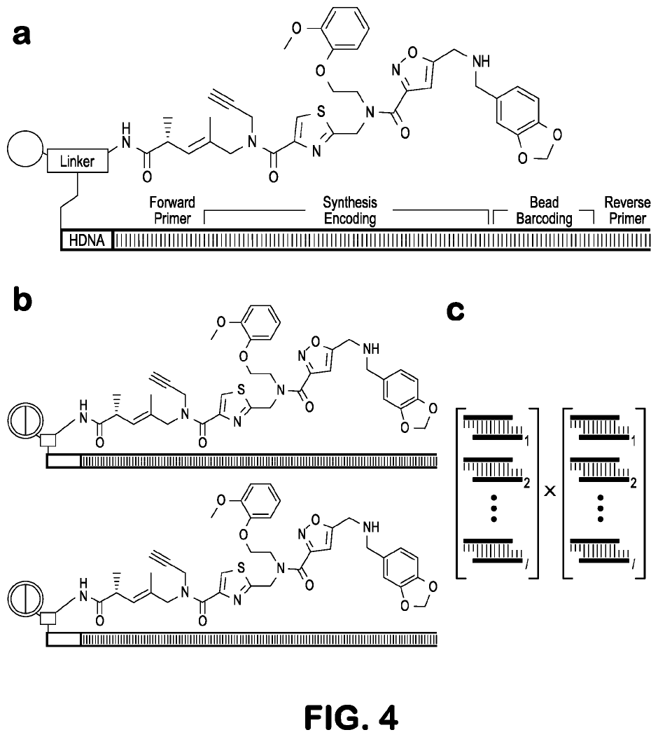

In one embodiment, disclosed herein is a polynucleotide-encoded chemical library comprising a plurality of compound library beads, wherein the beads comprise: a chemical moiety comprising a compound library member; a polynucleotide moiety comprising: an oligonucleotide encoding the compound library member structure, and a barcode identifying the bead; and a linking moiety, linking the chemical moiety to the polynucleotide moiety. In one embodiment, the barcode identifying the bead is an oligonucleotide. In one embodiment, the polynucleotide and/or oligonucleotide are a DNA oligonucleotide. In one embodiment, the polynucleotide encoded chemical library comprises two or more bead members having the identical compound library member, identical oligonucleotide encoding the compound library member structure, but different barcodes identifying each bead. In one embodiment, the presence of identical compound library members in more than one bead while having different barcodes identifying each bead enables discriminating between the two or more beads carrying the same compound library member. In one embodiment, the barcode identifying the bead comprises an oligonucleotide having a length of 2 to 20 nucleosides. In one embodiment, the barcode identifying the bead comprises an oligonucleotide having a length of 2 to 50 nucleotides. In one embodiment, the polynucleotide moiety is synthesized in solid phase on the beads. In one embodiment, the oligonucleotide encoding the compound library member is ligated in parallel with the compound library member synthesis. In one embodiment, following barcoding of the bead, polynucleotide encoded split-and-pool synthesis proceeds with alternating steps of monomer coupling followed by oligonucleotide ligation based encoding. In one embodiment, the oligonucleotide sequences encoding the compound library member structure and/or identifying the bead are thermodynamically optimized. In one embodiment, the oligonucleotide sequences encoding the compound library member structure and/or identifying the bead possess Hamming string distances ≥3. In one embodiment, the oligonucleotide sequences encoding the compound library member and/or identifying the bead has a total read length <100 bases for facile sequencing. In one embodiment, the oligonucleotide sequences encoding the compound library member structure and/or identifying the bead are thermodynamically optimized. In one embodiment, the linker comprises a chromophore. In one embodiment, the chromophore is coumarin. In one embodiment, the linker comprises a chemical moiety that enhances mass spectrometric ionization efficiency. In one embodiment, the chemical moiety is arginine. In one embodiment, the linker comprises an alkyne for copper catalyzed azide-alkyne cycloaddition click chemistry. In one embodiment, the barcode identifying the bead enables removal of false positive hits. In one embodiment, the polynucleotide sequencing data obtained after a screen reveal both the structure of the hit compounds and provide hit reproducibility data that rejects false positives. In one embodiment, the rejection of false positives justifies further downstream re-synthesis and functional characterization. In one embodiment, the bead count correlates with molecular properties such as potency and/or selectivity. In one embodiment, the bead displays oligomer, barcode region, and structure encoding region as shown in FIG. 1. In one embodiment, the bead displays oligomer, barcode region, and structure encoding region as shown in FIG. 4.

In another embodiment, disclosed herein is a method of combinatorial screening comprising the steps of: (i) incubating a fluorescently labeled protein with a polynucleotide-encoded chemical library comprising a plurality of bead members, wherein the beads comprise a chemical moiety comprising a compound library member, a polynucleotide moiety comprising an oligonucleotide encoding the compound library member structure, and a barcode identifying the bead, and a linking moiety, linking the chemical moiety to the polynucleotide moiety; (ii) washing the beads to remove excess unbound protein; (iii) sorting and detecting the beads that have bound to the labeled protein; (iv) amplifying the polynucleotide encoding tag sequences of the hit beads using PCR; (v) sequencing the polynucleotide moiety; and (vi) identifying the hit compound library members' structures based on the sequence of the polynucleotide encoding the compound. In one embodiment, the barcode identifying the bead is an oligonucleotide. In one embodiment, the polynucleotide and/or oligonucleotide are DNA oligonucleotides. In one embodiment, the binding data is deemed to be accurate if more than one bead containing identical compound library members is identified and/or more than one bead-specific barcode identifies the same compound library member.

In one embodiment, disclosed herein is a kit for combinatorial screening comprising: a polynucleotide encoded chemical library comprising one or more bead members, wherein the beads comprise a chemical moiety comprising a compound library member, a polynucleotide moiety comprising an oligonucleotide encoding the compound library member, and a barcode identifying the bead and a linking moiety, linking the chemical moiety to the polynucleotide moiety; and instruction for using the kit for combinatorial screening. In one embodiment, the instruction for using the kit is a printed instruction, video instruction, and/or audio instruction.

In one embodiment, disclosed herein is a method of yielding a diagnostic panel of molecules for a disease comprising: (i) providing a sample from a patient afflicted with the disease, and sample from a control individual who is not afflicted with the disease; (ii) screening the samples against a polynucleotide encoded chemical library; (iii) utilizing a fluorescent tag to label hit compound beads for fluorescence-activated cell sorting (FACS); (iv) deep sequencing all hits to determine the structure of the hit compounds and each hit's occurrence frequency; (v) pruning disease-afflicted hits from the unafflicted control hits; and (vi) resynthesizing the patient hits to yield a diagnostic panel for the disease. In one embodiment, the disease is active tuberculosis (ATB). In one embodiment, the control individual is someone who has noninfectious/latent TB (LTB). In one embodiment, the sample is a serum sample. In one embodiment, the fluorescent tag is anti-human IgG. In one embodiment, the diagnostic panel of drug molecules comprises thermally stable and economically produced small molecules.

In one embodiment, disclosed herein is a device, comprising a chemical moiety linked to a polynucleotide moiety, wherein the polynucleotide moiety comprises a barcode region and a binding region. In one embodiment, the binding region binds with specificity to a compound library member. In one embodiment, the barcode region indicates a specific bead. In one embodiment, the device is a screening device.

As further described herein, in one embodiment, the encoding region directly specifies the synthesis history of the bead (i.e. the sequence of reaction conditions that the bead experienced), and thereby indirectly the structure of the compound on the bead. Occasionally, the synthesis history may yield unanticipated products. These unanticipated products may also be important in target binding during screening, identifying the bead as a hit. Subsequent re-synthesis and purification would then putatively uncover the identity of the side product. In one embodiment, as will be readily appreciated by those skilled in the art, the bead barcoding approach is not restricted to identical compound structures. As one example, beads may display identical encoding regions, but different bead-specific barcodes. In one embodiment, whether the encoding region is encoding a synthesis history, chemical structure, or any other information is immaterial—the bead-specific barcode disclosed herein allows the differentiation of authentic/true positive hits (a single encoding region is observed with many bead-specific barcodes) from false positives (a single encoding region is observed with one bead-specific barcode) using the high-throughput sequencing data to differentiate reproducible hits from those only observed a single time.

Further, as will be readily appreciated by those skilled in the art, the hit identification as described herein is not restricted to FACS screening. Screening is fundamentally a way of separating beads with desirable properties from those that do not. FACS analysis of fluorescently-labeled beads is one methodology. The same could be accomplished with a magnetic selection, by sorting droplets, or by observing activity surrounding beads splayed out in an ordered or disordered array. Outputs from all screens/selections of DNA-encoded combinatorial bead libraries can be amplified, sequenced, and subjected to the sequencing-based hit authentication/prioritization described herein.

The kit disclosed herein is useful for practicing the inventive method of barcoding beads used in combinatorial screening. The kit is an assemblage of materials or components, including at least one of the inventive compositions. Thus, in some embodiments the kit contains a composition including chemical library comprising members which comprise a chemical moiety comprising a compound library member, a DNA moiety comprising: an oligonucleotide encoding the compound library member structure, and an oligonucleotide identifying the bead (barcode), and a linking moiety, linking the chemical moiety to the DNA moiety, as described above.

The exact nature of the components configured in the inventive kit depends on its intended purpose. For example, some embodiments are configured for the purpose of combinatorial screening of drug molecule candidates. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to yield a diagnostic panel of molecules for a disease. Optionally, the kit also contains other useful components, such as, diluents, buffers, pharmaceutically acceptable carriers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in scientific research industry. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing barcoded beads for combinatorial screening. The packaging material generally has an external label which indicates the contents and/or purpose of the kit and/or its components.

Embodiments of the present disclosure are further described in the following examples. The examples are merely illustrative and do not in any way limit the scope of the invention as claimed.

### EXAMPLES

### Example 1

### DNA Bared Bead Specific Barcoding

FIG. 1 illustrates one embodiment of the DNA based bead specific barcoding, wherein two encoding positions comprise the “barcoding region.” The barcoding region was constructed by splitting the bead sample into four ligation reactions containing one of four different magenta sequences. The samples were pooled, then split again into four ligation reactions now each containing one of four different gray sequences. The total number of barcodes generated in this fashion was 16 (4²). Each bead thus displayed many copies of 1 out of the 16 different generated barcodes. After split-and-pool ligation barcoding, DNA-encoded split-and-pool synthesis proceeded with alternating steps of monomer coupling (“diversity elements”) followed by oligonucleotide ligation-based encoding (DNA elements in the encoding region).

In order to reduce bead-specific barcoding to practice, the inventors started a DNA-encoded solid-phase synthesis (DESPS) using bifunctional resin prepared as described in MacConnell et al. ACS Comb. Sci. 2015, and incorporated by reference herein in its entirety. The inventors used a 10-digit numeric identifier code in order to describe different oligonucleotide sequences. Briefly, each oligonucleotide sequence received a 4-digit code. The first digit described a coding set (either set 1 or set 2; set 1 contained 30 unique coding sequences and set 2 contained 38 unique coding sequences). The second digit described the position in the tag. As an example, in FIG. 1, there were 10 coding positions in the DNA, which were enumerated 1, 2, 3, 4, 5, 6, 7, 8, 9, A. Set 1 sequences were used only at positions 1, 3, 5, 7, and 9. Set 2 sequences were used only at positions 2, 4, 6, 8, and A. Finally, the last 2 digits index unique coding sequences: 01, 02, 03, 04 . . . 30 for set 1. Concatenating these digits gave a unique code that specified the coding sequence set, the position within the coding tag, and the coding sequence. For example, oligonucleotide code 2405 was a set 2 sequence used at position 4 and it was sequence “05” from the set 2 group of sequences. To barcode the resin, 8 set 1 position 1 sequences and 10 set 2 position 2 sequences were used for split-and-pool ligation as outlined above to generate B=80=10×8 unique bead-specific barcodes. This was referred to as the “barcoded resin.”

Next, the inventors used the barcoded resin to synthesize a DNA-encoded compound library following the dual-scale approach described MacConnell et al. The library diversity featured 84 different structures at 3 diversification positions, yielding a 84³=592,704-member library. The library chemistry was encoded using 84 different combinations each of 13XX24XX, 15XX26XX, and 17XX28XX. During library synthesis at the third diversification position, a small portion of the resin was coupled to control ligands biotin or iminobiotin. Biotin was assigned coding sequence 17072801 and iminobiotin was assigned coding sequence 17072802. These two control ligand pools were maintained as separate wells, their encoding tags finished, and maintained as separate positive control stocks (i.e. they were not mixed back into the library) for subsequent screening. The final 2 coding positions (19XX2AXX) were assigned library ID codes, and not used for any bead or structure decoding. The bead-barcoded encoded library was subjected to quality control (QC) by removing all 160-μm QC particles, isolating individual particles for PCR amplification, sequencing and mass spectrometric analysis to correlate sequence-predicted exact mass and observed exact mass (MacConnell el al. 2015). The 10-μm particles were retained for high-throughput screening by FACS.

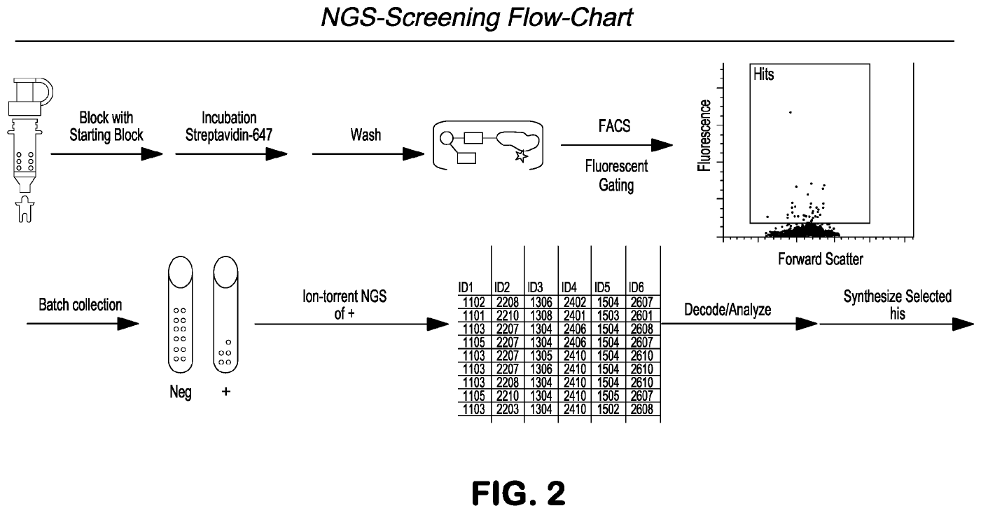

Aliquots of library (˜3 MM beads) were used to develop a FACS-based high-throughput screening protocol (FIG. 2). To the library was added 300 encoded biotinylated (Ser. No. 17/072,801) beads and 200 encoded iminobiotinylated (Ser. No. 17/072,802) beads. The library was incubated in Starting Block proprietary protein mixture to prevent non-specific adsorption. The library was washed, combined with streptavidin-Alexafluor647 (SA647, 100 nM+50% Starting Block in PBS-T buffer), incubated (1 h, RT), and washed three times (PBS-T buffer). The aliquot was loaded into the FACS instrument (FACSJazz, BD Bioscienccs) and sorted (λex=640 nm, λem=660 nm). The analysis covered 2.7 MM events corresponding to a compound redundancy of 4.6 and yielding 2,579 “hits” that exceeded the background fluorescence threshold. A second screen was executed on a second aliquot of the resin. The analysis covered 2.9 MM events corresponding to a compound redundancy of 4.9, and yielded 3,125 hits. These hits were subjected to a second round of sorting into high- and low-fluorescence bins of 242 and 1743 hits, respectively. After screening, each the DNA encoding tags on the beads of each hit pool were amplified in PCR and sequenced using a pyrosequencing-based high-throughput sequencer (Ion Proton, Invitrogen), yielding a sequence file for structure decoding.

The sequence file was then fed into an informatics workflow that the inventors developed specifically for these types of data sets. Briefly, the sequences were read into the script and pattern matched to the reference sequence:

(SEQ ID NO: 1)
“ATGGNNNNNNNNTCANNNNNNNNGTTNNNNNNNNCTANNNNNNNNTTCNN
NNNNNNCGCNNNNNNNNGTANNNNNNNNTGGNNNNNNNNTCTNNNNNNNNA
AGNNNNNNNNGCCT″

Fixed sequences represented the constant overhangs used for cohesive end ligation during encoding. “NNNNNNNN” were the 8-mer coding regions.

Matched sequencing reads were next corrected for sequencing errors and decoded to numeric identifier strings. The genetic language design distributed the sequences in set 1 and set 2 such that all members were maximally genetically distinct (Hamming distance >2). Thus, sequence analysis could tolerate one sequencing error in each coding region and still assign a correct coding sequence. After error correction, reads were aggregated to unique sequences, rank-ordered by the number of reads per unique sequence, j sequences with the highest number of reads (where j is the number of hit beads sequenced in the pool) were further split into numeric identifiers using the overhangs. Overhang ATGG preceded position 1, TCA precedes position 2, and so on. The sequence “ATGGACGAGATT” (SEQ ID NO:2) was decoded to 1103 because ACGAGATT was a member of sequence set 1, the ATGG overhang signified position 1 in the coding tag, and ACGAGATT was sequence #03 of set 1. These identifiers together encode a unique bead barcode, molecular structure encoding tag, and library ID tag: “1109220813022403150726081707280819112A02” is an example of such a compound library member identifier.

The compound library member identifiers were used to count individual biotinylated and iminobiotinylated positive control hits from each of the ˜3 MM bead screens. All sequences containing either 17072801 or 17072802 identifiers were tabulated to obtain the number of observed positive control ligand beads. The first screen yielded 209 (out of ˜300) hits encoding biotin and 126 (out of ˜200) hits encoding iminobiotin. The second screen similarly yielded 224 biotin hits and 149 iminobiotin hits. Because the control ligands were appended to bona fide library members, the total number of sequences encoding for either biotin or iminobiotin was 80×84×84×1 (80 bead specific barcodes, 84 position 1 sequences, 84 position 2 sequences, 1 position 3 sequence=564,840. This gave the error in counting (E, see equation above). The number of biotinylated hits was 7.6% and the E for the iminobiotinylated hits was 3.4%.

The remaining hits that were not biotin or iminobiotin were further analyzed, using the bead-specific barcodes to count the number of instances each structure was observed in the hit pool. Six redundantly isolated structures of interest emerged from the data set. For the purposes of this disclosure, as an illustrative example, the discovery of compound 2 is described. Compound 2's numeric identifier was 130624081510260517102805 with bead barcoding and library ID stripped from the sequence. Without bead-specific barcoding, this would be the only sequence information describing compound 2, and it would have registered as a single hit in screen 1 and a single hit in screen 2. However, the sequencing data revealed 32 instances of this identifier, 16 unique bead-specific barcodes in screen 1 (“11102206” “11012208” “11072201” “11092209” “11092205” “11032202” “11082208” “11032209” “11042207” “11072207” “11082206” “11012202” “11062210” “11042201” “11062203” “11012204”) and 16 unique bead-specific barcodes in screen 2 (“11072205” “11102208” “11082208” “11092203” “11102201” “11102202” “11042205” “11092205” “11012205” “11032206” “11102207” “11042201” “11012206” “11092204” “11042209” “11062201”). Compounds 1, 3, 4, 5, and 6 shared redundancy with 2, and were progressed to re-synthesis and validation.

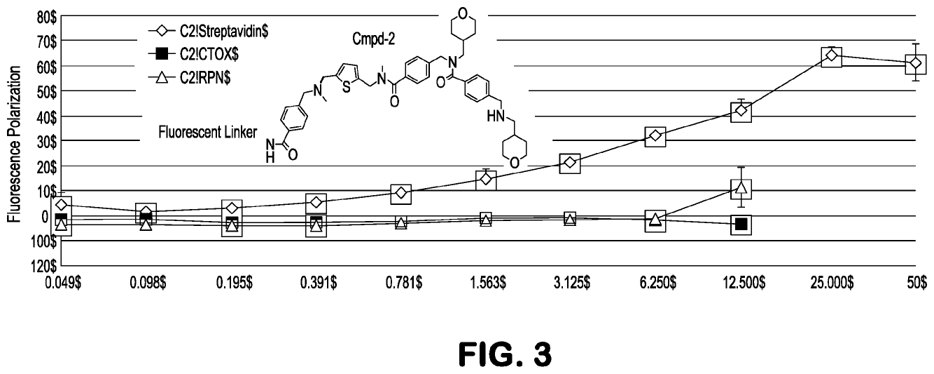

Compounds were prepared with a fluorescein label, diluted (10 nM) in PBS-T buffer, and incubated with streptavidin target at varying concentration. Fluorescence anisotropy was used to determine the binding constant (FIG. 3, ˜12 μM). Compound 2 binds streptavidin selectively compared to other protein targets currently under screening and is competitive with the endogenous streptavidin ligand, biotin.

The other five compounds exhibited similar affinity binding of target, though with off-target binding interactions. Though they are not leads that would garner additional interest, they nonetheless bound the target of the screen.

### Example 2

### Utility

Split-and-pool solid-phase synthesis provides an extremely efficient route to large compound bead libraries for screening. Screening such bead libraries typically entails incubating the library with a labeled target, washing unbound target, harvesting labeled library members (the hit compounds), determining the structures of the hits, then resynthesizing the hits for functional characterization. While the first steps of this process (synthesis and screening) are extremely efficient in terms of throughput, high false positive rates (sometimes >90%!) during screening pose a commercially disabling drawback because resynthesis and functional screening (hit compound validation) require a significant investment of manpower. Pursuing false positives virtually negates all synthesis and screening throughput advantages.

Given the throughput limitations of resynthesis and high false positive rate, implementing strategies that discriminate true hits from false positives is uniquely enabling. One approach that proved highly effective entailed observing the same compound as a hit on different beads from redundant libraries. In fact, it was possible to discriminate true hits from false positives by observing the same compound as a hit on as few as 2 different beads (Doran et al 2014). Similar observations prompted the single-molecule counting strategy that is used to discriminate true hits from noise in DNA-encoded library screening (Clark et al 2009).

In one embodiment, this present disclosure provides another novel, effective, and easy to use method for discriminating true hits from false positives. The present disclosure provides a method of DNA barcoding each bead such that the DNA sequence could be used not only to decode the compound library member structure but also to discriminate identical compounds present on multiple different beads. Unlike conventional DNA-encoded libraries where simple randomized oligonucleotides could be used for single-molecule counting, the present method required generating many copies of a barcode on each bead. The split-and-pool ligation barcoding strategy described here enabled bead counting with accuracy limited only by the number of unique barcodes generated. In the example of FIG. 1, 16 barcodes are possible. The probability that two identical compounds inhabiting two distinct beads yet displaying identical barcodes is 1/16, which represented the false negative rate (the DNA sequences in both barcoding region and encoding regions are identical and therefore would appear as a single bead in DNA sequence data). It can be shown that E, the probability of incorrectly counting N distinct beads each displaying the same compound and labeled with one of B possible distinct barcodes is:

In the 16-barcode example, the probability of incorrectly counting N=2 beads is 6%. The probability of incorrectly counting N=5 beads is 50%. This error can be minimized by increasing B, which is trivial given that the barcodes are generated by split-and-pool ligation; conducting 10 different ligations at 3 different positions would yield 1,000 different barcodes, and would reduce the error rate in counting N=5 beads to 1%.

### Example 3

### Advantages

The high false positive rates in bead-based compound library screening are disabling in a commercial setting where manpower to conduct resynthesis is prohibitively expensive. Manually separating, sequencing (either by mass spectrometry or Sanger DNA sequencing) and counting beads similarly compromise the process. Barcoding the beads in a manner that allows sequence-based bead counting eliminates all manual steps in bead hit identification. All hit beads can be pooled, amplified in one pot, and the resulting templates analyzed in a single next-generation DNA sequencing experiment. The sequence data reveal the compound structures and provide hit reproducibility data that reject false positives, justifying further downstream resynthesis and functional characterization.

### Example 4

### High-Throughput Identification of DNA-Encoded IgG Ligands that Distinguish Active and Latent Mycobacterium Tuberculosis Infections

The detection of specific lgG populations in the circulating repertoire forms the basis of numerous immunological diagnostics such as the ELISA. However, the discovery of IgGs with diagnostic potential usually follows identification of their cognate antigens. The complexity of this task grows as the number of potential antigens increases from a relatively small immunoproteome (e.g. HIV) to the much larger spaces of pathogenic bacteria or the human proteome. Further, many diseases occur in multiple clinically distinct states, such as viral or bacterial latency, requiring a dissection of antigen identity, IgG response, and clinical manifestation.

Mycobacterium tuberculosis (Mtb) infection status, for example, can be one of two classifications. Differentiating these two statuses a major priority of the World Health Organization in the surveillance and treatment of the disease. The latent, noninfectious state (LTB) is defined by granulomatous lesions that encase the pathogen. In the active and infectious state (ATB), rapidly dividing bacilli invade pulmonary and other tissues, replicate, and eventually cause symptoms. Neither current point-of-care tests (tuberculin skin test) nor more advanced assays (interferon gamma release, PCR) can differentiate status. The stark differences between the pathogen's LTB and ATB metabolic states suggest that the host immunological response may provide the most discriminatory signals. Protein microarray data point to a small collection of candidate antigens—mostly comprising membrane-associated and secreted proteins (e.g. ESAT-6, CFP-10, Ag85)—that could generate the desired differential response. Extensive investigations of these and other antigens' suitability as TB serological diagnostics have ensued, however, no single antigen yields appropriate diagnostic sensitivity and specificity. Furthermore, ongoing studies increasingly highlight the importance and prevalence of TB-specific post-translational modifications (PTMs) particularly on secreted antigens, ultimately necessitating mycobacterial antigen production and thereby raising scale-up and stability challenges for diagnostic development. Serial native antigen evaluation thus poses a daunting combinatorial and logistical challenge.

It is possible to circumvent both up-front antigen selection biases and production bottlenecks by combinatorially querying IgG repertoires corresponding to known patient statuses. Differentially probing a protein microarray that displayed a rich sampling of the Mtb proteome led to an experimental definition of its immunoproteome, the subset of Mtb immunodominant proteins. Phage display epitope libraries are used to pan lgG repertoires for peptide antigen mimetics (“mimotopes”) in many disease contexts, including the identification of antigenic proteins in TB. However, peptides are susceptible to proteolytic degradation and costly to produce at scale. It has been shown that combinatorial libraries of N-substituted oligoglycines (“peptoids”) and other non-natural oligomers can source IgG ligands (“epitope surrogates”) specific for Alzheimer's disease, neuromyelitis optica, chronic lymphocytic leukemia, and type 1 diabetes (T1D). Epitope surrogates can serve as affinity reagents for selective purification of the disease-specific IgGs and subsequent native antigen identification. For example, an epitope surrogate discovered from a screen of T1D patient sera ultimately identified peripherin as a major T1D autoantigen. The T1D-specific antibodies recognize only a highly phosphorylated, dimeric form of the protein, suggesting that native antigens of the disease-specific antibodies are unlikely to be “vanilla” peptides or recombinantly-expressed proteins. Synthetic epitope surrogates not only serendipitously mimic chemical functionality beyond the space of the 20 biogenic amino acids, but are potentially advantageous for diagnostics because they resist proteolytic degradation, are economically synthesized, and do not require refrigeration-all qualities of diagnostics that are amenable to resource-limited and point-of-care settings.

The discovery of epitope surrogates from combinatorial libraries of synthetic molecules is currently a manual and tedious process. A one-bead-one-compound (OBOC) library of molecules (i.e., each bead displays many copies of a single molecule) displayed on 90-μm TentaGel beads is incubated in control sera, beads displaying compounds that bind to control antibodies are visualized with a fluorescent anti-lgG secondary antibody, and manually removed. The remaining library is incubated in case serum and the process is repeated to isolate putative ligands to antibodies unique to, or highly enriched in, the case. The chemical structure of the hit ligands is then elucidated by mass spectrometry (MS) one bead at a time. Due to the low throughput of manual bead picking and MS structure elucidation, it is not feasible to build consensus structures as in phage display, where next-generation sequencing (NGS)-based analysis can now detail the phylogenctic history of an antigen's discovery.

DNA-encoded small molecule libraries (DELs) have provided an elegant approach to marrying the power of genetic information storage and retrieval with access to diverse chemotypes via chemical synthesis. Encoded combinatorial synthesis entails coupling a nucleic acid encoding step with each chemical synthesis step, and after selection-type separation of target ligands, NGS analysis is used to decode the structures of all hits. Potent ligands have resulted from DEL selections against a variety of purified targets, but it stands to reason that such combinatorial libraries could be even more useful in a phenotypic assay, where the target identity is unknown. In one embodiment, in this disclosure, the inventors have demonstrated the use of DNA-encoded combinatorial libraries of non-natural oligomers for unbiased IgG repertoire screening, and NGS analysis to discover statistically significantly represented hit structures and structurally homologous families of ATB-specific epitope surrogates.

### Example 4

### Library Synthesis

A solid-phase DNA-encoded combinatorial library was synthesized using peptide couplings and the sub-monomer method employed to construct peptoids and similar compounds. The 448 k-member library featured diversity at three positions (Post, Pos₂, Pos₃) in both the main chain scaffolding and side chains using a variety of building block (BB) types. Pos₁ contained a collection of amino acids (both stereochemical configurations) and diverse submonomer-type BBs (haloacids and amines for halide displacement). Pos₂ and Pos₃ contained only submonomer-type BBs. The library was synthesized on a dual-scale mixture of 10-μm screening beads and 160-μm quality control (QC) beads, the latter doped at a low level (QC:screening=1:30,000). After synthesis, the QC beads were harvested, the DNA-encoding tags of single QC beads were amplified, sequenced, and decoded to yield the bead's synthesis history and predicted compound structure. MALDI-TOF MS analysis of the corresponding resin-cleaved compound was then compared to the encoding-predicted structure mass. The spectra of 19/20 QC bead compounds were consistent with the DNA-encoded structures, which collectively contained at least one instance of 34/60 BBs used for library synthesis.

### Example 5

### FACS-Based High-Throughput Screening

ATB-selective serum IgG-binding ligands were identified using FACS-based high-throughput screening. Both single-color and two-color strategies were explored. The one-color screens were performed by incubating ˜10 copies of the library (˜5×10⁶ beads) with pooled serum samples acquired from 10 ATB patients. Another ˜10 copies was incubated with a mixture of sera acquired from 10 LTB patients and 10 “normal control” (NC) individuals who had not been exposed to Mtb, comprising the “NCL” pool. After washing, the beads were incubated with a secondary detection IgG (Alexa Fluor 647 anti-human IgG) to label serum lgG-binding hit compound beads for collection by FACS. The screen yielded 6297 ATB hit beads and 8579 NCL hit beads. A control screen for library beads that bind the secondary detection IgG in the absence of serum was also performed, yielding 447 beads.

The same ATB and NCL serum pools were used for a two-color screen. Addition of a secondary detection mFab (Alexa Fluor 488 anti-human mFab, mFab488) to the NCL serum labeled the NCL IgGs in one color while addition of a differently labeled secondary detection mFab (Alexa Fluor 647 anti-human mFab, mFab647) to the ATB serum labeled ATB IgGs with the second color. The pre-labeled sera were mixed and incubated with DNA encoded library beads (5×10⁶). Beads with high 660-nm fluorescence (ATB serum) and low 530-nm fluorescence (NCL serum) were isolated by FACS (723 beads. The hit bead collection DNA-encoding tags of each screen were separately amplified, sequenced, and decoded to generate lists of candidate NCL and ATB IgG ligands.

### Example 6

### Encoding Tag Analysis and Pan-Library Structure-Activity Relationship Profile

NGS analysis of the hit bead collection amplicons generated lists of hit sequences for decoding based on a modified encoding tag structure (FIG. 4a). The synthesis encoding tag structure was expanded to accommodate eight (8) encoding regions, the first six positions used to encode chemical synthesis and the final two positions used to assign bead-specific barcodes. Bead-specific barcodes were used to differentiate redundant hits (i.e. identical compounds observed as hits on different beads, FIG. 4b) and tabulate hit occurrence frequency for each screen. The four TB screens (single-color secondary detection IgG only, single-color ATB, single-color NCL, and two-color ATB/NCL) generated 2086 unique encoding sequences. Single-color data were pruned of all synthesis encoding sequences that occurred with only one bead-specific barcode, after which 792 ATB hit sequences remained. All hit sequences that also appeared in the secondary detection IgG only and NCL single-color screens were eliminated, leaving 351 ATB hit sequences. The two-color screen, which internally controlled NCL and non-specific lgG binding, generated 88 unique synthesis encoding sequences that occurred with more than one bead-specific barcode, 85 of which did not appear in either the secondary detection lgG only or NCL single-color screens. Of the reduced ATB single-color and two-color hit sequence sets, 36 occurred in both screening modes.

The relative occurrence of each monomer in the one- and two-color ATB hit sequence pool in conjunction with the hit occurrence frequency derived from bead-specific barcodes guided the selection of hits for resynthesis. The pan-library structure-activity relationship data, shown as a plot of the position-dependent occurrence frequency of each monomer (% observed) in comparison with its occurrence frequency in a random sample of the library, illuminated highly enriched structural features of each screening hit collection. In addition to this “bottom-up” analysis of structure conservation among hits, a “top-down” census of hits that occurred with the highest frequency between both screening pools was also conducted. Of the 36 hit sequences observed in both ATB screens, 27 were observed ≥5 times and the top 10 hits were observed ≥8 times. Hit sequences that occurred with high frequency and contained more frequently observed monomers were prioritized for resynthesis. This included 18 of the 36 hit sequences observed in both screening modes and 3 hit sequences derived from highly enriched monomers. The 21 representative hit sequences were clustered into four thematic synthesis histories: (1) heterocycle haloacid or 4-(bromomethyl)-benzoic acid BBs in all 3 positions, (2) heterocycle haloacid BBs in Pos₂ and Pos₃ with Pos₃ N-(3-aminopropyl)-2-pyrrolidinone displacement, (3) either stereochemistry chloropentenoic acid BB in Pos₁, and (4) pyridine-containing BBs in Pos₁.

The encoded synthesis histories of the 21 representative hits were reproduced on a larger scale with a C-terminal cysteine. These products were purified and appended to resin via thioalkylation for validation using a Luminex-like assay previously developed in our laboratory. Serum IgG binding assay results of 16/21 hit sequences indicated ATB-selective binding over NCL binding for at least one product at the screening serum concentration (1000 μg/mL, LOD>3, p=0.005) and 13/21 yielded at least one product that maintained ATB-selective binding at lower serum concentration (250 μg/mL, LOD>3, p=0.005). Reproducing the synthesis histories coding N-(3-aminopropyl)-2-pyrrolidinone in Pos₃ yielded both the expected product and a side product, both of which selectively bound ATB serum IgGs. NMR analysis of the isoxazole N-(3-aminopropyl)-2-pyrolidinone Pos₃ monomer supported assignment of a side product structure that results from an acid-catalyzed cyclization and concomitant loss of water. Resynthesis of sequences coding for pyridine-containing Pos₁ monomer produced beads that were red and did not selectively bind ATB serum IgGs. These false positives were likely identified by FACS sorting due to their high intrinsic fluorescence. Resynthesis of all hit sequences with heterocycle haloacid or 4-(bromomethyl)-benzoic acid BBs in Posₜ, Pos₂ and Pos₃ yielded the expected major product, and selectively bound ATB serum IgGs at both serum concentrations (0.25 and 1 mg/mL). The expected products of sequences coding for chloropentenoic acid BBs in Posₜ selectively bound ATB serum IgGs at [serum]=1 mg/mL (7/10 hits) and [serum]=0.25 mg/mL (4/10 hits).

### Example 7

### Patient-Specific Binding Validation

Hit structures that validated with pooled serum samples used for library screening were next tested for binding to serum IgG repertoires of individual patients. The “discovery” patient sample set comprised those serum samples used for library screening (10 ATB, 10 LTB, 10 NC), and the “test” patient sample set comprised all other samples that were not used for library screening (40 ATB. 44 LTB, 11 NC). Competition binding with soluble ligand was then assayed for individuals that scored binding above the a threshold. This competition experiment was critical because some serum samples contained antibodies that exhibited high non-specific adsorption. If less than 50% of the original signal was competed by excess soluble molecule, it was treated as a negative result. Overall, NC and LTB patient-specific analyses across discovery and test sets responded minimally in the set of ligands analyzed. NC patient-specific serum IgG binding assays of 15 resynthesized hit compounds were only positive for binding in three ligands. Only one LTB discovery set patient responded to a ligand bound, but more signals were observed in the larger test set. Two LTB test set patients responded specifically to multiple ligands. Of the LTB test, 7/44 samples responded specifically to at least one ligand. 9/10 ATB discovery set patients responded specifically to at least one ligand though binding was not evenly distributed between patients and ligands. For example, five different ligands responded similarly in six ATB discovery patients. Likewise, another ATB discovery patient responded to 8/15 validation hits. Overall 11/40 ATB test patients responded specifically to at least one ligand.

The competition binding data guided the selection of 4 ligands that maximally sampled the ATB discovery set patient samples. 6/10 ATB discovery set serum samples contained IgGs that bind selectively to one of the four structures with >50% soluble ligand competition. No significant antibody binding to these compounds was observed in the LTB discovery samples, whereas antibodies in two of the normal control samples were retained by two hits. However, in these cases, less than 50% of the signal was competed. All NC and LTB discovery patient samples bound with <50% soluble ligand completion. The panel exhibited 60% sensitivity, 100% specificity, 100% positive predictive value (PPV), and 83.3% negative predictive value (NPV) for all discovery set samples. The same panel exhibited 30% sensitivity, 96% specificity, 83% PPV, and 70% NPV for all discovery and test set samples.

### Example 8

### Antigen Discovery

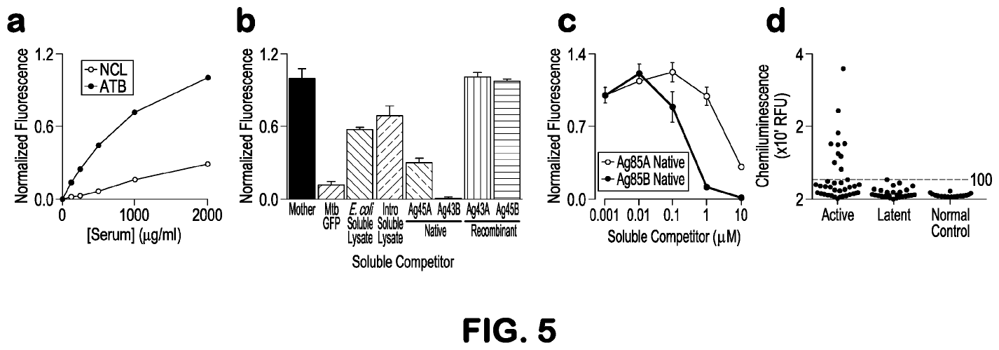

Competition binding analysis of pooled ATB serum samples with a ligand 2-B and a variety of Mtb-associated proteins was performed in an attempt to identify the native antigen that 2-B mimics. Ligand 2-B exhibited strong and selective ATB serum IgG binding (FIG. Sa). Culture filtrate proteins (CFP) derived from several hypervirulent Mtb strains (HN878, CDC1551. H37Rv) competed efficiently for binding whereas the E. coli and Mtb lysates competed weakly (FIG. 5b), illustrating that the antigen might be secreted. Further examination of several secreted proteins purified from Mtb revealed that Ag85A and Ag85B compete strongly with 2-B for binding ATB serum IgGs. Competition titration analysis of Ag85A and Ag85B with 2-B showed that Ag85B bound ATB IgGs ˜10-fold better than Ag85A (FIG. 5c). From this data, the inventors concluded that compound 2-B mimics an epitope displayed on the native Ag85B. All other purified native and recombinant Mtb proteins, including the recombinant forms of Ag85A and Ag85B, did not compete with 2-B for ATB serum binding. Western analysis of native Ag85B, H37Rv culture filtrate proteins, and CDC1551 culture filtrate proteins using either antibodies that were affinity purified from ATB patient serum on a column functionalized with compound 2-B or anti-Ag85 complex indicated that 2-B-specific antibodies specifically react with Ag8SB, again supporting the hypothesis that 2-B is an epitope surrogate of Ag85B. Immobilized native Ag85B used in an ELISA experiment analogous to the patient-specific epitope surrogate experiments yielded a diagnostic sensitivity of 22% and specificity of 100% for the entire collection of discovery and test patient serum samples (FIG. 5d).

### Example 9

### DNA-Encoded Combinatorial Library

Using a DNA-encoded combinatorial library for differentially probing the IgG repertoire of case and control serum samples introduced numerous advantages for epitope surrogate discovery related to the orders of magnitude increases in throughput that FACS and NGS enable. The small (10 μm) TentaGel beads employed for library construction both facilitated large library synthesis (each gram of resin contains 1000-fold more 10-μm beads than conventional 90-μm beads) and the use of FACS-based screening, which quantitatively analyzes and collects several thousand compound beads per second. This represented a vast improvement over manual bead picking, which is slow and, absent custom screening technology, subjective. The greatly enhanced throughput of NGS-based structure elucidation uniquely provided rapid and deep analysis of hit structures, critical for matching the throughput of FACS. These expansive data not only revealed hit structures, but insight into structural features important for IgG binding. For example, in the screen described here, the data argue that conformational constraint is important for lgG binding, in agreement with previous screens of non-DNA-encoded oligomer libraries. The library is ˜6% peptoid (less conformationally constrained) in Pos₂ and Pos₃, but this motif appeared in only 0.9% of the hit structures.

DNA-encoded synthesis also enabled the use of structurally diverse BBs that otherwise confound MS-based structure elucidation. Incorporation of heterocycle-containing haloacids and chioropentenoic acid BBs conformationally constrained the main chain scaffold, potentially mitigating the entropic penalty of binding associated with the “floppier” peptoid chemotype. The MS fragmentation spectra of oligomers composed of these BBs were complex, however, and almost untenable in a library. The hit structure families of this screen almost ubiquitously featured such BBs, resulting in highly heterogeneous main chain scaffolds. Similarly, imperfect or unanticipated reactivity can generate cryptic signals that compromise MS analysis. DNA-encoded synthesis readily facilitated the elucidation of products arising from such reactivities as well. For example, some compounds with a terminal N-(3-aminopropyl)-2-pyrrolidinone moiety unexpectedly rearranged upon release from the beads with some rearrangement products performing better than the parent compound. The −18 m/z rearrangement product, which for some hits was the major product, would have been nearly impossible to deduce by MS alone, but was readily rationalized upon inspection and reproduction of the DNA-encoded synthesis history. DNA-encoded synthesis may begin to relax decades-old yield and purity constraints of library synthesis reactions as these and other results from DNA-encoded combinatorial libraries are establishing that chemistry can be “error-prone” as long as the encoded synthesis history is reproducibility at scale and preserves sufficient PCR-viable DNA for decoding.

In one embodiment, the bead-specific barcodes disclosed herein mark a significant advance in encoding that is uniquely critical to OBOC screening. High false discovery rates are common and problematic for on-bead screening, but observing a hit multiple times on distinct beads (redundancy) signals authentic target binding. In previous language design, identical compounds present on multiple beads would be indistinguishable by sequencing. The present disclosure provides bead-specific barcodes to count such redundant hits, which occur at frequencies in these experiments requiring few distinct barcodes for accurate counting. The probability of correctly counting redundant hit beads using bead-specific barcodes is identical to the classic birthday problem: “how many students must be in a class to guarantee that at least two students share a birthday?” Here, the barcodes are the birthdays, the beads are the students, and “birthday twins” are beads that will be miscounted by serendipitously sharing identical bead-specific barcodes. The probability, P. of N beads displaying unique bead-specific barcodes selected from B total barcodes and therefore being correctly counted is:

$$P = \frac{B !}{{B}^{N} ( B - N ) !} = \frac{( \begin{matrix}B \\ N\end{matrix} )}{{B}^{N}}$$

For this study, P=88% for N=5 (the typical number of library copies observed in a FACS experiment) and B=80 bead-specific barcodes. As barcodes are combinatorially generated, it is straightforward to access very large B either by using more sequence modules per position, reassigning synthesis encoding positions to bead barcoding, or further expanding the number of positions. However, the modest B of this study was sufficient to develop a top-down structure census that, combined with bottom-up consensus analysis, formed the foundation of a highly effective hit prioritization strategy and striking validation success rate (16/21).

The DNA-encoded library screen efficiently identified small molecules that specifically bound to ATB discovery patient serum-derived IgGs and not those present in the NCL discovery set, and binding specificity translated well to the test sets. Of the validated hit structures, all but one bound specifically to at least one ATB discovery set patient's serum IgGs. The LTB and NC discovery set patient sera responses were also gratifyingly clear of positive responses. No patients in the NC test set responded positively to the validated ligands, however two LTB test patients responded positively and specifically to numerous ligands in a pattern that is strikingly similar to six ATB discovery patients. A likely explanation for this is that these LTB patients could be undergoing reactivation, and therefore serologically appear as if they are ATB. Alternatively, it is possible that some ligands may not discriminate well between ATB and LTB.

One high-priority hit family generated unanticipated side products that selectively bound ATB serum IgGs. Competition binding analysis implicated ligand 2-B, a representative of the family, as an epitope surrogate of the immunodominant Mtb secreted protein Ag85B. The antigen 85 complex (Ag85A, Ag85B, Ag85C) is abundantly secreted during an ATB infection. The Ag85 proteins are diacylglycerol acyltransferases that mediate the incorporation of mycolic acid into the pathogen's cell wall and binding to fibronectin, both of which are critical for infection of and proliferation in macrophages. That 2-B mimics an epitope of Ag85B is consistent with the antigen's expression in ATB, however, 2-B exhibited no binding competition with Ag85B expressed recombinantly in E. coli. Differences in protein folding between expression hosts or the presence of host-specific PTMs could explain this observation. Further proteomic analysis will clarify this observation, though it is not strictly necessary to elucidate the nature of the antigen mimicry for the purposes of diagnostic development.

The diagnostic sensitivity of an ELISA using native Ag85B as a non-specifically immobilized antigen was low, consistent with previous work and this study. Ag85B, when used as the sole biomarker for serological diagnosis, yields a spread sensitivities (4-84%). In our hands, the native antigen is also not very sensitive, though quite specific. Notably, however, the immobilized antigen identified an entirely different population of ATB patients; neither discovery nor test ATB patient sera that were positive for 2-B binding responded positively in the immobilized Ag85B ELISA. Non-specifically immobilized antigens can occlude the epitope that the small-molecule is mimicking. This does not rule out Ag85B as a diagnostic antigen or viable target for mimicry as a surrogate. On the contrary, Ag85B, when part of a “TB antigen cocktail,” yielded a 98% sensitive diagnostic, in line with both the previously observed spread of diagnostic sensitivities for all TB antigens studied in isolation and our observations of enhanced sensitivity using the epitope surrogate panel. Further expansion of this panel is underway to generate an analogous small molecule cocktail that is far more economical to produce and thermally stable.

In one embodiment, the inventors have found that while both one- and two-color strategies contributed to the hit structures, the two-color approach was more selective (and experimentally more efficient). One-color screening hits are derived from subtraction of hits that occur in two control screens (the NCL patient serum and secondary detection antibody only) from those observed in the case screen (ATB). The two-color screen obviated the need for separate control screens by detecting NCL-selective ligands and ATB-selective ligands in separate color channels, while non-selective ligands (including ligands of the secondary mFab antibody) populate the diagonal. Furthermore, this approach was more stringent as ˜10-fold fewer hits are observed directly as selective ligands in the two-color experiment versus deriving selectivity by comparison of multiple one-color screens. Regardless of screening format, however, several ATB discovery patients' sera dominated the IgG binding profile of the library. Screening combinatorially pooled case samples in conjunction with a small subset of single-patient case samples (e.g. ATB) generated an abbreviated survey of each ligand candidate's diagnostic sensitivity and specificity prior to resynthesis, providing even deeper predictive statistics to guide the selection of epitope surrogates for constructing an optimally sensitive panel.

### Example 10

### Materials and Methods

Materials Sources.

All reagents were obtained from Sigma Aldrich (St. Louis. Mo.) unless otherwise specified. N,N′-diisopropylcarbodiimide (DIC, Acros Organics, Fair Lawn, N.J.), 1-hydroxy-7-azabenzotriazole (HOAt), N,N-diisopropylethylamine (DIEA, Thermo Fisher Scientific. Waltham, Mass.). 2,4,6-trimethylpyridine (Oxyma, Sigma Aldrich), N-α-Fmoc-Arg(Pbf)-OH (Anaspec, Fremont, Calif.), N-α-Fmoc-Gly-OH (Anaspec, Fremont, Calif.), N-α-Peg2-OH (Chiral Polyamines), cyclopropylmethylamine (AK Scientific, Union City, Calif.), 2-(2′-methoxy)phenoxyethylamine (AK Scientific), 2-phenoxyethylamine (AK Scientific), m-(trifluoromethoxy)benzylamine (AK Scientific), 4-cyanobenzylamine (AK Scientific), 4-bromobenzylamine (AK Scientific), homopiperonylamine (AK Scientific), neopentylamine (TCI America, Portland, Oreg.), methallylamine (Chem-Impex, Wood Dale, Ill.), 2-cyclohexylethylamine (Alfa Aesar, Ward Hill, Mass.), 2-(2-aminoethyl)thiophene (Alfa Aesar. Ward Hill. Mass.), Fmoc-D-alanine (EMD Millipore, Billerica. Mass.), Fmoc-D-leucine (EMD Millipore), Fmoc-D-phenylalanine (EMD Millipore), Fmoc-L-norvaline (EMD Millipore), Fmoc-L-norleucine (EMD Millipore), N-α-Fmoc-β-cyclohexyl-L-alanine (EMD Millipore), Fmoc-homo-L-phenylalanine (EMD Millipore), dimethylformamide (DMF, Thermo Fisher Scientific), dichloromethane (DCM, Thermo Fisher Scientific), acetic anhydride, trifluoroacetic acid (TEA), triisopropylsilane (TIPS), diethyl ether, dimethyl sulfoxide (DMSO), α-cyano-4-hydroxycinnamic acid (HCCA), formic acid, phenol, acetonitrile (HPLC grade, Thermo Fisher Scientific), H₂O (HPLC grade, Thermo Fisher Scientific), triethylammonium acetate (TEAA, 2 M, Life Technologies), L(+)-ascorbic acid (Acros Organics), copper (II) sulfate (CuSO4), ethylenediaminetetraacetic acid (EDTA), ammonium citrate dibasic, sodium hydroxide, ethanol, sodium citrate dibasic, Taq DNA polymerase (Taq, New England Biolabs, Ipswich, Mass.), 2′-deoxyribonucleoside triphosphates (dNTP, set of dATP, dTTP, dGTP, dCTP, New England Biolabs), agarose, and T4 DNA ligase (New England Biolabs), polyclonal anti-Mycobacterium tuberculosis antigen 85 complex (FbpA/FbpB/FbpC; antiserum, Rabbit, BE Resources, Mannassas, Va.) were used as provided.

Solvents used in solid-phase synthesis were dried over molecular sieves (3 Å, 3.2 mm pellets). Heterocyclic haloacid and chloropentenoic acid BBs were prepared as previously described. Tris[(1-benzyl-1H-1,2,3-triazol-4-yl)methyl]amine (TBTA) was recrystallized three times in t-BuOH/H₂O (1:1). Oligonucleotides (Integrated DNA Technologies, Inc., Coralville, Iowa) were obtained as desalted lyophilate and used without additional purification.

The Mycobacterium tuberculosis culture filtrate proteins were obtained through BEI Resources, NIAID, NIH: Strain CDC1551, NR-14826; Strain HN878, NR-14827; Strain H37Rv, NR-14825. The Mycobacterium tuberculosis whole cell lysates were obtained through BEI Resources, NIAID, NIH: Strain CDC1551, NR-14823; Strain HN878, NR-14824; Strain Indo-Oceanic T17X, NR-36496; Strain East African Indian 91_0079, NR-36497; Strain H37Rv. NR-14822. The Mycobacterium tuberculosis purified native proteins were obtained through BEI Resources, NIAID, NIH: Ag85A (Rv3804c), Strain H37Rv, NR-14856; Ag85B (Gene Rv1886c), Strain H37Rv, NR-14857; Ag85C (Gene Rv0129c), Strain H37Rv, NR-14858; Ag85 Complex, Strain H37Rv, NR-14855; α-Crystallin (Gene Rv2031c), Strain H37Rv, NR-14860; GroES (Gene Rv3418c), Strain H37Rv, NR-14861; MPT32/Apa (Gene Rv1860), Strain H37Rv, NR-14862; PstS1 (Gene Rv0934, Non-Acylated), Strain H37Rv, NR-14859. The Mycobacterium tuberculosis recombinant protein reference standards were obtained through BEI Resources, NIAID, NIH: Ag85A, NR-49427; Ag85B, NR-14870; CFP-10, NR-49425; ESAT-6, NR-14868.1; HspX, NR-31384.

The Anti-Ag85 antibody was obtained through BET Resources, NIAID, NIH: Polyclonal Anti-Mycobacterium tuberculosis Antigen 85 Complex (FbpA/FbpB/FbpC; Genes Rv3804c, Rv1886c, Rv0129c) (antiserum, Rabbit), NR-13800.

Buffers.

10×Bis-Tris propane ligation buffer (BTPLB, 500 mM NaCl, 100 mM MgCl2, 10 mM ATP, 0.2% Tween 20, 100 mM Bis-Tris, pH 7.6), Bis-Tris propane wash buffer (BTPWB, 50 mM NaCl, 0.04% Tween 20, 10 mM Bis-Tris, pH 7.6), 1×GC-PCR buffer (IX PCR buffer, 8% DMSO, 1 M betaine), saline-sodium citrate hybridization buffer (SSC, 150 mM NaCl, 15 mM citrate, 1% SDS, pH 7.6), 10×PCR buffer (2 mM each dNTP, 15 mM MgCl2, 500 mM KCl, 100 mM Tris, pH 8.3) were prepared in DI H₂O.

Bifunctional Resin Synthesis and Characterization.

Azido headpiece DNA (HDNA) was prepared using techniques readily known in the art. Linker synthesis on mixed TentaGel rink amide resin (160 μm, 0.41 mmol/g, 4 mg, Rapp-Polymere) and amino resin (10 μm, 0.23 mmol/g, 30 mg, Rapp-Polymere) were mixed and transferred to a fritted spin-column (Mobil Classic, large filter, 10-μm pore size) and swelled in DMF (1 h, RT). Linker synthesis proceeded via iterative cycles of solid phase peptide or peptoid synthesis. Each amino acid coupling cycle consisted of: (1) Fmoc-deprotection (20% piperidine in DMF, 500 μL, 1×5 min, 1×10 min, 8 rpm, RT); (2) N-α-Fmoc-amino acid (90 μmol, 500 μL DMF) activation with DIC/Oxyma/DIEA (90/90/180 μmol) and incubation (2 min, RT); (3) addition of activated N-α-Fmoc-amino acid to resin and incubation (1 h, 37° C., 8 rpm). Following each deprotection and coupling step, resin was washed using a vacuum manifold (DMF 1×5 mL, DCM, 1×5 mL, DMF 1×5 mL). Each peptoid incorporation cycle consisted of: (1) bromoacetic acid (90 μmol, 500 μL DMF) activation with DIC/Oxyma/DIEA (90/90/180 μmol) and incubation (2 min, RT); (2) addition of activated bromoacetic acid to resin, incubation (1 h, 37° C., 8 rpm), and washing (DMF 1×5 mL, DCM, 1×5 mL, DMF 1×5 mL); (3) haloacid displacement (I M amine, 500 μL DMF, 2 h, 37° C., 8 rpm), and washing (DMF 1×5 mL, DCM, 1×5 mL, DMF 1×5 mL). N-α-Fmoc-Arg(Pbf)-OH, N-α-Fmoc-Arg(Pbf)-OH, bromoacetic acid, 4-bromobenzylamine, N-α-Fmoc-Gly-OH, bromoacetic acid, propargylglycine, and N-α-Fmoc-PEG₂-OH were coupled sequentially as described above. Mixed-scale bifunctional-HDNA library resin was prepared and characterized as readily known in the art.

DNA-Encoded Solid-Phase Combinatorial Library Synthesis.

Mixed-scale bifunctional-HDNA library resin was aliquotted to a fritted spin column, washed (DMF, 1×500 μL), Fmoc-deprotected (20% piperidine in DMF, 500 μL, 1×5 min, 1×10 min, 8 rpm, RT), washed (DMF 1×5 mL; DCM, 1×5 mL; DMF 1×5 mL), transferred to a 5 mL Eppendorf tube, and resuspended (DMF, 3.75 mL). Resin was split (50 μg 160 μm, 2 nmol; 0.4 mg 10 μm, 90 nmol) into 75 wells of a pre-wet (DCM, 100 μL) filtration microplate (Millipore MultiScrcen Solvinert 0.45 μm Hydrophobic PTFE, EMD Millipore, Billerica, Mass.). Library synthesis proceeded through iterative cycles of monomer synthesis, encoding oligonucleotide ligation, and Fmoc-deprotection.

Monomer Synthesis.

Monomer coupling consisted of either (1) acylation with an N-α-Fmoc amino acid or (2) acylation using a haloacid and subsequent halide displacement with a primary amine. N-α-Fmoc amino acid and haloacids (12 μmol, DMF, 150 μL) were activated with DIC/Oxyma/TMP (75/12/12 μmol. 5 min, RT), then added to the appropriate wells of the filtration microplate. Plates were covered with adhesive foil (VWR International, Radnor, Pa.) and incubated with agitation (1 h, 37° C., 800 rpm). Following incubation, mixtures were drained and resin was washed (DMF, 3×150 μL; DCM, 1×150 μL; DMF, 1×150 μL). Amines (1 M, DMF, 150 μL) or DMF (150 μL) were added to wells previously reacted with haloacid and N-α-Fmoc amino acid respectively, covered with adhesive foil, and incubated with agitation (3 h, 37° C., 800 rpm). Following incubation, mixtures were drained and resin was washed (DMF, 3×150 μL; DCM, 1×150 μL; DMF, 1×150 μL; 1:1 DMF:BTPWB, 3×150 μL; BTPWB, 2×150 μL), resuspended (BTPWB, 1×150 μL), covered with adhesive foil, incubated (30 min, RT, 800 rpm), resuspended in BTPWB (100 μL) while the encoding oligonucleotide ligation mixtures were prepared (˜30 min, RT), and washed (BTPTL, 1×100 μL).

Ligation of 0001, ≈11XX and ≈22XX Encoding Oligonucleotides.

An encoding oligonucleotide ligation mixture containing ≈0001[±] (120 nmol), and T4 DNA ligase (22500 U) in 1.35×BTPLB (11 mL) was prepared and aliquoted into all plate wells (100 μL). OP stocks of ≈11XX[±] (1.2 nmol, 20 μL) and ≈22XX[±] (1.2 nmol, 20 μL) were then added to the appropriate wells, the plate was sealed with adhesive foil, and incubated with agitation (4 h, RT, 800 rpm). Resin was washed (BTPWB, 3×150 μL; 1:1 DMF:BTPWB, 3×150 μL; DMF, 3×150 μL), resuspended (DMF, 150 μL) and incubated (16 h, RT, 800 rpm). Resin was pooled in a fritted spin column, washed (DMF, 1×500 μL), Fmoc was removed (20% piperidine in DMF, 500 μL, 1×5 min, 1×10 min, 8 rpm, RT), washed (DMF, 4×500 μL; DCM, 2×500 μL; DMF 3×500 μL), transferred to a clean centrifuge tube, and resuspended (DMF, 4 mL). Resin was split (50 μg 160 μm, 2 nmol; 0.38 mg 10 μm, 86 nmol) into 80 wells of a pre-wet (DCM, 100 μL) filtration microplate for monomer coupling.

Ligation of ≈13XX and ≈24XX Encoding Oligonucleotides.

An encoding oligonucleotide ligation mixture containing T4 DNA ligase (15000 U) in BTPLB was prepared and aliquoted into all plate wells (110 μL). OP stocks of ≈13XX[±] (1.2 nmol, 20 μL) and ≈24XX[±] (1.2 nmol, 20 μL) were then added to the appropriate wells, the plate was sealed with adhesive foil, and incubated with agitation (12 h, RT, 800 rpm). Resin was pooled in a fritted spin column, washed (DMF, 4×500 μL; DCM, 2×500 μL; DMF 3×500 μL), transferred to a clean centrifuge tube, and resuspended (DMF, 4 mL). Resin was split (50 μg 160 μm, 2 nmol; 0.38 mg 10 μm, 86 nmol) into 80 wells of a pre-wet (DCM, 100 μL) filtration microplate for monomer coupling.

Ligation of ≈15XX and ≈26XX Encoding Oligonucleotides.

An encoding oligonucleotide ligation mixture containing T4 DNA ligase (15000 U) in BTPLB was prepared and aliquoted into all plate wells (110 μL, 148 U T4 DNA ligase. OP stocks of ≈15XX[±] (1.2 nmol, 20 μL) and ≈26XX[±] (1.2 nmol, 20 μL) were then added to the appropriate wells, the plate was sealed with adhesive foil, and incubated with agitation (12 h, RT, 800 rpm). Resin was pooled in a fritted spin column, washed (DMF, 4×500 μL; DCM, 2×500 μL; DMF 3×500 μL), transferred to a 5-mL microcentrifuge tube, and resuspended (DMF, 4 mL).

Ligation of Barcoding ≈17XX and ≈18xx, and 0901 Encoding Oligonucleotides.

Resin was split (50 μg 160 μm, 2 nmol; 0.38 mg 10 μm, 86 nmol) into 80 wells of a pre-wet (DCM, 100 μL) filtration microplate, washed (1×150 μL; 1:1 DMF:BTPWB, 3×150 μL; BTPWB, 2×150 μL), resuspended (BTPWB, 1×150 μL), covered with adhesive foil, incubated with agitation (30 min, RT, 800 rpm), resuspended in BTPWB (100 μL) while the encoding oligonucleotide ligation mixtures were prepared (˜30 min, RT), and washed (BTPLB, 1×100 μL). An encoding oligonucleotide ligation mixture containing ≈0901[±] (120 nmol), and T4 DNA ligase (22,500 U) in 1.35×BTPLB (11 mL) was prepared and aliquoted into all plate wells (100 μL). OP stocks of ≈17XX[±] (1.2 nmol, 20 μL) and ≈28XX[±] (1.2 nmol, 20 μL) were then added to the appropriate wells, the plate was sealed with adhesive foil, and incubated with agitation (4 h, RT, 800 rpm). Resin was washed (BTPWB, 3×150 μL; 1:1 DMF:BTPWB, 3×150 μL, DMF, 3×150 μL), resuspended (DMF, 150 μL) and incubated (16 h, RT, 800 rpm). Resin was pooled in a fritted spin column and washed (DMF, 1×500 μL)

DNA-Encoded Library Quality Control.

Resin was pooled in a fritted spin column, and washed (DMF, 4×500 μL; DCM, 2×500 μL; DMF 3×500 μL), resuspended (DMF, 500 μL), and sonicated (30 s). The 160-μm beads were removed by filtration (150-μm mesh, CellTrics 150 μm, Partec), collected, and stored (DMF, 4° C.). The eluted 10-μm resin was collected into a fritted spin column and resuspended (DMF, 450 μL). An aliquot of 10-μm resin (0.5 mg) was transferred to a 1.5-mL tube, washed (BTPWB, 4×500 μL) with centrifugation (6000 ref), and resuspended (BTPWB, 500 μL). The bead concentration was determined by hemocytometer and normalized (1.2 beads/μL, BTPWB). An aliquot of 160-μm library resin was transferred to a 1.5-mL microcentrifuge tube and washed (BTPWB, 5×500 μL; 1×500 μL, 1 h, RT).

qPCR Analysis.

qPCR matrix contained Taq DNA Polymerase (0.05 U/μL), oligonucleotide primers 5′-GCCGCCCAGTCCTGCTCGCTTCGCTAC-3′ (SEQ ID NO:3) and 5′-/5AmMC6/GTGGCACAACAACTGGCGGGCAAAC-3′ (SEQ ID NO:4) (0.3 μM each), SYBR Green (0.2×, Life Technologies), and GC-PCR buffer (1×). Single 160-μm resin beads (1 μL, BTPWB) were added to separate amplification wells containing qPCR matrix (20 μL, 22 replicates). 10-μm library beads (1 μL, 1.2 beads/μL, BTPWB) were added to separate amplification wells containing qPCR matrix (20 μL, 227 replicates). Supernatant for each resin sample (1 μL) was added to separate amplification wells (20 μL, 3 replicates). Template standard solutions (1 μL, 100 amol, 10 amol, 1 amol, 100 zmol, 10 zmol, 1 zmol, 100 ymol, and 10 ymol in BTPWB) were added to separate amplification reactions (20 μL). Reactions were thermally cycled (96° C., 10 s; [95° C., 8s; 72° C., 24 s]×30 cycles; C1000 Touch Thermal Cycler, Bio-Rad, Hercules, Calif.) with fluorescence monitoring (channel 4, CFX96 Real-Time System, Bio-Rad) and quantitated (CFX Manager, Version 3.1, Bio-Rad, baseline subtracted). The number of amplifiable tags per bead was calculated by dividing the qPCR result by the number of beads per well (confirmed using a stereo zoom microscope).

Amplification and Sequencing.

qPCR matrix contained Taq DNA Polymerase (0.05 U/μL), oligonucleotide primers 5′-GCCGCCCAGTCCTGCTCGCTTCGCTAC-3′ (SEQ ID NO:3) and 5′-/5AmMC6/GTGGCACAACAACTGGCGGGCAAAC-3′ (SEQ ID NO:4) (0.3 μM each). SYBR Green (0.1×, Life Technologies), and PCR buffer (IX). Single 160-μm beads (1 μL, BTPWB) were added to separate amplification wells containing qPCR matrix (20 μL, 33 replicates). Resin supernatant (1 μL) was added to separate amplification wells (20 μL, 3 replicates). Template standard solutions (1 μL, 100 amol, 10 amol, 1 amol, 100 zmol, 10 zmol, 1 zmol, 100 ymol, and 10 ymol in BTPWB) were added to separate amplification reactions (20 μL). Reactions were thermally cycled (95° C., 15 s; [72° C., 30 s]×26 cycles) with fluorescence monitoring. Single 160-μm resin beads were retrieved via pipet from PCR plate wells and deposited into a 96-well filtration microplate (MeOH, 150 μL). Each 160-μm library bead PCR sample (6 μL) was purified by native PAGE (6% 1×TBE, 6W, 30 min). Gel slices containing 145-nt DNA products were excised and eluted in C&S buffer (300 μL, 18 h, RT, 8 rpm). PCR matrix containing Taq DNA Polymerase (0.05 U/μL), oligonucleotide primers 5′-GTTTTCCCAGTCACGAC-3′ (0.3 μM) and 5′-GTGGCACAACAACTG-3′ (SEQ ID NO:10) (0.28 μM) and 5′-CGCCAGGGTTTTCCCAGTCACGACCAACCACCCAAACCACAAACCCAAACCCCA AACCCAACACACAACAACAGCCGCCCAGTCCTGCTCGCTTCGCTAC-3′ (SEQ ID NO:9) (0.02 μM, FOX primer), and GC-PCR buffer (IX). PAGE-purified PCR templates (2 μL) were added to separate amplification reactions (20 μL) and thermally cycled ([95° C., 20 s; 52° C., 15 s; 72° C., 20 s]×25 cycles). PCR products were purified (QIAquick PCR purification kit, QIAGEN, Valencia, Calif.) and sequenced using the M13F(-41) primer (GeneWiz, South Plainfield, N.J.). Sequencing reads were trimmed to remove all called bases prior to the opening primer sequence (5′-GCCGCCCAGTCCTGCTCGCTTCGCTAC-3′) (SEQ ID NO:3). Sequences were aligned to a degenerate reference sequence (5′-GCCGCCCAGTCCTGCTC-GCTTCGCTACATGGNNNNNNNNTCANNNNNNNNGTTNNNNNNNNCTANNNNNN NNTTCNNNNNNNNCGCNNNNNNNGTFNNNNNNNNCTANNNNNNTNNGCCTGTT TGCCCGCCAGTTGTTGTGCCAC-3′) (SEQ ID NO:7) and the encoding regions (5′-NNNNNNNN-3) (SEQ ID NO:8) were matched to the structure-identifier lookup table to assign the synthesis history for each compound.

Resin Cleavage and MALDI-TOF MS Analysis.

Individual 160-μm beads were washed (DI H₂O, 3×150 μL; 100 mM triethylammonium bicarbonate pH 8.5, 2×150 μL; DMF, 4×150 μL), incubated (15 min, RT), transferred in DMF (5 μL), into separate microplate wells, washed (DMF, 3×150 μL; DCM, 3×150 μL) and dried in a centrifugal evaporator (15 min, 40° C.). Cleavage cocktail (90% TFA, 5% DCM, 5% TIPS, 50 μL) was added to dried single 160-μm library bead samples, incubated (1 h, RT), and dried in vacuo. Residue was resuspended (50% ACN, 0.1% TFA in H2O, 7 μL) and an aliquot (1 μL) cospotted onto a MALDI-TOF MS target plate with HCCA matrix solution (see above), dried, and analyzed via MALDI-TOFiTOF MS/MS (4800 Plus MALDI TOF/TOF Analyzer, Applied Biosystems, Foster City, Calif.).

FACS Based Screening.

All patient serum samples were obtained from Gerhard Walzl of Stellenbosch University and included three classes of patients: normal control, latent TB infection, and active TB infection. A pool of serum composed of equal volumes of 10 normal control and 10 latent TB infection patients was prepared (600 μg/mL in PBS StartingBlock, NCL pool). A pool of serum composed of equal volumes of 10 active TB infection patients was prepared (600 μg/mL in PBS StartingBlock, ATB pool).

Single-Color Library Screening Sample Preparation.

Library beads (˜5×10⁶ per screen) were exchanged (TBST, 500 μL), the supernatant was decanted, and the resin was resuspended in PBS StartingBlock (1 mL), and incubated (1 h, 4° C.) to yield a pre-blocked library aliquot. NCL pool (1 mL), ATB pool (I mL), and PBS StartingBlock (1 mL) were each added to separate pre-blocked library aliquots. Samples were incubated with rotation (18 h, 4° C., 8 rpm). Each aliquot was washed (TBST, 3×1 mL). Goat Anti-Human IgG (H+L) Alexa Fluor 647 conjugate was diluted (1:200 in PBS StartingBlock), added to each library aliquot (1 mL) and incubated with rotation (2 h, 4° C., 8 rpm). The beads were washed (TBST, 3×1 mL) and resuspended (TBST, 1.2 mL) for FACS analysis.

Two-Color Library Screening Sample Preparation.

The NCL pool (600 μg/mL, 250 μL) was mixed with Alexa Fluor 488 Anti-Human mFab conjugate (mFab488, 800 μg/mL, 250 μL, Jackson ImmunoResearch, West Grove, Pa.). The ATB pool (600 μg/mL, 250 μL) was mixed with Alexa Fluor 647 Anti-Human mFab conjugate (mFab647, 800 μg/mL, 250 μL, Jackson ImmunoResearch, West Grove, Pa.). The mixtures were incubated with rotation (30 min, RT, 8 rpm). Human lgG agarose beads (125 μL) were washed (PBS, 3×1 mL), added to the serum-mFab mixtures, and incubated with rotation (10 min, RT, 8 rpm). The mixture was filtered (Multiscreen HTS 96 well filter-bottom plate. EMD Millipore Corporation, Darmstadt, Germany) into a clean 96-well plate to yield mFab-labeled serum. The mFab488-labeled NCL pool (500 μL) was combined with the mFab647-labeled ATB pool (500 μL). The mixture of labeled serum was incubated with a pre-blocked library aliquot, washed, and prepared for sorting as described above.

Facs Analysis.

Samples were sorted (BD FACS Jazz, BD Biosciences, San Jose, Calif.) after calibration (Accudrop and Sphere rainbow standards, BD Biosciences). Forward and side scatter were used to define a gate for the single-bead population. A fluorescence intensity threshold (30,000 RFU, 660-nm channel) was set for single-color screening samples (secondary antibody only, NCL and ATB) to activate sorting. Prior to two-color screens, an aliquot of the two-color library screening sample (100 k beads) was used to adjust laser intensities (488 nm and 640 nm), and detector voltages (530- and 660-nm channels) such that the signals from each channel were ˜1:1. Fluorescence intensity thresholds (20,000-40,000 RFU along a line equal to ⅔ of the 660-nm channel intensity, 530-nm channel; 30000, 660-nm channel) were set to activate sorting.

NGS Sample Preparation.

Beads were transferred from the FACS collection tube to a clean centrifuge tube (0.2 mL) and supernatant reduced (t₀ ˜5 μL). qPCR matrix contained Taq DNA polymerase (0.05 U/μL), oligonucleotide primers 5′-GCCGCCCAGTCCTGCTCGCTTCGCTAC-3′ (SEQ ID NO:3) and 5′-/5AmMC6/GTGGCACAACAACTGGCGGGCAAAC-3′ (SEQ ID NO:4) (0.3 μM each), SYBR Green (0.2×, Life Technologies), DMSO (8%), betaine (1 M), MgCl₂ (1 mM) and PCR buffer (1×). qPCR matrix was added to 0.2 mL tubes (20 μL). Template standard solutions (1 μL, 100 amol, 10 amol, 1 amol, 100 zmol, 10 zmol, 1 zmol, 100 ymol, and 10 ymol) were added to separate amplification reactions (20 μL). Reactions were thermally cycled ([95° C., 8 s; 72° C., 24 s]×30 cycles). Samples were centrifuged briefly. The amplicon-containing supernatants were transferred to clean tubes, and diluted (1:10000 in BTPWB). PCR matrix contained Taq DNA Polymerase (0.05 U/μL), oligonucleotide primer 5′-CCTCTCTCTATGGGCAGTCGGTGATGTGGCAACTGGCGGGCAAAC-3′ (SEQ ID NO:5) (0.3 μM), SYBR Green (0.05×, Life Technologies) DMSO (6%), betaine (1 M), MgCl₂(1 mM) and PCR buffer (1×). Amplicon dilution (2 μL) and a corresponding NGS barcode oligonucleotide primer, 5′-CCATCTCATCCCTGCGTGTCTCCGACTCAGCTAAGGTAACGATGCCGCCCAGTCC TGCTCGCTFCGCTAC-3′ (SEQ ID NO:6) (12 pmol), were added to separate amplification wells (40 μL). Reactions were thermally cycled ([95° C., 8 s; 72° C., 16 s]×18 cycles). Barcoded amplicon samples (5 μL) were purified by native PAGE (6%, 1×TBE, 4 W, 30 min) with SYBR Gold staining (Life Technologies, Inc.). Gel slices containing 211-nt DNA products were excised, samples combined in a tube (0.5 mL) punctured at the bottom using a syringe needle (18 gauge) and the sample was centrifuged (5 min, 10,000 RCF). Dl H₂O was added (100 μL), the sample was incubated (overnight, RT, 8 rpm), centrifuged (5 min, 10,000 RCF), and the supernatant removed to a clean tube. An aliquot was used for standard NGS sample preparation and sequencing (Ion Proton, Life Technologies, Inc.).

NGS Decoding and Structure Elucidation.

IonTorent fastq files for each screening sample set were imported into R⁵, each sequence was matched to the 8-position reference sequence

(SEQ ID NO: 1)
″ATGGNNNNNNNNTCANNNNNNNNGTTNNNNNNNNCTANNNNNNNNTTCNN
NNNNNNCGCNNNNNNNNGTTNNNNNNNNCTANNNNNNNNGCCT,″

and sequences were trimmed based on the degenerate reference sequence. All NNNNNNNN encoding sequences were matched with the known encoding set (“TGGAAAGT”, “ACGGAGCA”, “TTGGAGTT”, “AAGGAGGT”, “AGAAAGCA”, “ACAGAACT”, “TAAGGAGT”. “ATGGGAGT”, “TGAAGGAA”, “TTGAGGAT”, “CCTCCTAA”, “AACCTCAA”, “AATCCCAT”, “AACCCTAC”, “ATCCTCTC”, “CATTTCAA”, “CGCCTTCA”, “CGTTCCTG”, “TTCTTCAT”, “TCCTCTTA”). Hamming distances were calculated for all non-matched NNNNNNNN encoding sequences. Those with Hamming distance=1 from a member of the known encoding set were replaced with the correct sequence. Any read containing an encoding sequence with Hamming distance >1 was removed. Identical sequences were aggregated as a single sequence and the number of reads the sequence was observed. Identifiers (1101-1110, 2201-2210, 1301-1310, 2401-2410, 1501-1510, 2601-2610, 1701-1710, 2801-2810) were assigned to each sequence. Sequences with read number less than 1×10⁻⁷ of the total reads that matched the degenerate reference were removed. The encoding sequences of positions 7 and 8 (the bead-specific barcodes of FIG. 4) were used to count sequences that were identical in positions 1-6 as redundant hits. Hit redundancy for each screening sample set was aggregated into a single data set, and identifiers were matched to the structure-identifier look up table to decode the corresponding hit structures.

Hit Resynthesis.

Oligomers were synthesized on Rink Amide MBHA resin (0.55 mmol/g, EMD Millipore Corporation). Resin (0.15 g, 0.0825 mmol) was swelled in DMF (2 h), Fmoc was removed (20% piperidine in DMF, 20 min, RT, 250 rpm) and washed (DMF, 3×5 mL). N-α-Fmoc-Cys(Trt)-OH (0.25 mmol), HBTU (0.25 mmol), HOBt (0.25 mmol), and DIEA (0.25 mmol) were combined in DMF (3 mL), added to resin, and the resin incubated with shaking (3 h, RT, 250 rpm). The resin was washed (DMF, 3×5 mL), Fmoc was removed (20% piperidine, 20 min, RT, 250 rpm) and the resin was washed (DMF, 3×5 mL). Fmoc-8-amino-3,6 dioxaoctanoic acid (0.25 mmol, Chiral Polyamines, Port St. Lucie, Fla.), HBTU (0.25 mmol), HOBt (0.25 mmol), and DIEA (0.25 mmol) were combined in DMF (3 mL), added to resin, and the resin incubated with shaking (3 h, RT, 250 rpm). The resin was washed (DMF, 3×5 mL), Fmoc was removed (20% piperidine, 20 min, RT, 8 rpm), and the resin was washed (DMF, 3×5 mL). Resin was acylated by preparing a solution of the appropriate acid monomers (80 mM), DIC (500 mM), Oxyma (80 mM), and TMP (80 mM) in DMF (3 mL), incubating (5 min, RT), then adding the activated carboxylic acid solutions to the resin and incubating with shaking (1 h, 37° C., 250 rpm). Resin was washed (DMF, 3×5 mL), the appropriate amine added (1 M in DMF, 1 mL), the resin incubated (3 h, 37° C., 250 rpm), and washed (DMF, 3×5 mL). Resin was washed (DCM, 3×5 mL) and dried using a vacuum manifold. Cleavage cocktail (95% TFA, 2.5% TIPS, 2.5% DI H₂O; 3 mL) was added to resin, and the resin incubated with shaking (2 h, RI, 250 rpm). Cleavage product was separated from resin and evaporated under argon, and the crude was precipitated with cold diethyl ether and pelleted by centrifugation. The pellet was resuspended (30% ACN in DI H₂O) and purified by reversed-phase HPLC with gradient elution (C18, 19 mm×250 mm, 10 μm, Waters XBridge BEH300, mobile phase A: ACN, mobile phase B: 0.1% TFA in H₂O; 10-90% A, 20 mL/min, 38 min) using a Waters 1525 binary HPLC with UV detection (220 nm, Waters 2487, Waters, Corp.). Product fractions were analyzed by MALDI-TOF MS (Applied Biosystems), the oligomers were lyophilized (VirTis SP Scientific), and stored dry.

FACS Hit Revalidation.

Bead Encoding and Ligand Immobilization.

TentaGel microspheres (100 mg, 10 μm, 0.23 mmol/g, Rapp Polymere) were encoded using Pacific Orange and Pacific Blue to create 24 fluorescently distinct populations. After dye encoding, the beads were washed (DMF, 4×1 mL), Fmoc was removed (20% piperidine in DMF, 2×500 μL, 15 min), and the resins washed (DMF, 4×1 mL). Fmoc-L-methionine, HBTU, HOBt, and DIEA (3 eq. each) were combined in DMF (1 mL), added to resins, and incubated with rotation (3 h, RT, 8 rpm). The resin was washed (DMF, 3×5 mL), Fmoc was removed (20% piperidine in DMF, 2×500 1 μL, 15 min each), and the resins were washed (DMF, 3×5 mL). Bromoacetic acid (2 M in DMF, 150 μL) and DIC (2.5 M in DMF, 150 μL) were added to resins, the resins were incubated with shaking (10 min, 37° C., 250 rpm) and washed (DMF, 6×1 mL). Purified oligomer solutions (3 mg/mL in 1:1 PBS:DMF, pH 7.4, 1 mL) were added to the respective fluorescently-encoded resin sample, and the resigns were incubated with rotation (overnight, RT, 8 rpm) and washed (DMF, 5×1 mL). BME (150 mM in 1 mL 1:1 PBS:DMF) was added to the resin. The resin was incubated (30 min, RT) and washed (DMF, 5×1 mL). The beads were transferred to a filtration microplate (MultiScreen Solvinert PTFE filter plate, EMD Millipore). The DMF was evacuated, resins were washed (DI H₂O, 10×300 μL) and incubated in DI H₂O (overnight, RT). An aliquot (˜100 μg) of each resin sample was removed, CNBr (30 mg/mL in 5:4:1 ACN:AcOH:DI H₂O, 25 μL) solution was added, and the resin incubated (overnight, RT). The CNBr solution was evaporated and the product dissolved (1:1 ACN:DI H₂O) and analyzed by MALDI-TOF MS (Applied Biosystems). The remaining resins were washed (TBST, 3×300 μL), transferred to a clean tube, and stored (4° C.).

Serum Binding Assays.

Encoded flow cytometry beads displaying the hit molecules of interest were pooled together in TBST (1 mL), sonicated (5 min), and filtered (40 μm, Cell Strainer Snap Cap, Falcon). Filtered aliquots (˜1 μg) were transferred to 96-well filtration microplate wells. PBS StartingBlock (100 μL) was added to each well and incubated (1 h, 4° C.). Discovery set serum pools were serially diluted in PBS StartingBlock (1, 0.5, 0.25, 0.125 mg/mL final serum concentrations). Individual patient serum samples were diluted in PBS StartingBlock (1 mg/mL final serum concentration). Each serum sample (90 μL) was combined with PBS (10 μL, 1 mM BME) to generate serum binding samples. Competitor oligomer solutions were prepared in PBS (100 μM competitor, 200 μM BME). Serum samples (90 μL) were combined with the appropriate competitor solution (10 μL) to generate oligomer competition serum binding samples.

Mycobacterium tuberculosis (Mtb) antigens (BEI Resources, Manassas, Va.) were prepared as a stock solution (5×) in PBS. Cell lysates were centrifuged (15 min, 15000 rpm). The culture filtrate proteins and soluble cell lysates were diluted (1.25 mg/mL in PBS). E. coli (DH5α, ThermoFisher Scientific, Waltham, Mass.) were grown in Luria broth (1 L) until OD600 ˜1.2. The cells were harvested by centrifugation (10000 rpm, 5 min), resuspended in PBS (20 mL, protease inhibitor cocktail tablet), lysed by sonication (30 s pulse, ×5), and the solution was clarified by centrifugation (15 min, 15000 rpm). The soluble lysate was diluted (1.25 mg/mL in PBS). Antigen competition serum binding samples were prepared by adding the previously described StartingBlock-diluted serum samples (80 μL) to antigen competitor stock (20 μL). Controls were prepared by combining diluted serum sample (80 μL) and PBS (20 μL). Once assembled, all sample types (serum binding, oligomer competition, antigen competition, and controls) were incubated (1 h, 4° C.).

The filtration microplate containing the flow cytometry beads was drained of StartingBlock by vacuum filtration. Prepared serum samples were added to the appropriate wells, and the microplate was incubated with shaking (overnight, 4° C., 250 rpm). Solution was drained from the filter plate and the beads were washed (TBST, 3×200 μL). Goat anti-human IgG (H+L) secondary antibody Alexa Fluor 647 conjugate (1:200 dilution in PBS, ThermoFisher Scientific) was added to each well and the plate was incubated with shaking (2 h, 4° C. 250 rpm). The beads were washed (TBST, 3×200 μL), resuspended in TBST (200 μL), and the contents of each well transferred to tubes for analysis (BD LSRII flow cytometer, BD Biosciences, San Jose, Calif.). The mean fluorescence intensity (MFI, λₑₘ=670 nm) of each encoded bead population was averaged across 2 independent experiments, and reported as the average MFI±σ of the two experiments. A≥3σ threshold was established using the MFI of all normal control patient serum samples. Patient serum samples that exhibited MFI≥3σ were scored as positive and all others as negative.

NMR Confirmation of a Proposed 2-B Side Product.

As shown below in scheme 1, solution-phase synthesis of the Pos₃ monomer of ligand 2-B proceeded via Dess-Martin oxidation (Dess-Martin periodinane, DCM) of the corresponding isoxazole alcohol (22 methyl 5-(hydroxymethyl)isoxazole-3-carboxylate 23), followed by coupling to N-(3-aminopropyl)-2-pyrrolidinone (Na₂SO₄, THF). Treatment of 24 under reducing conditions (NaCNBH₄, THF) produced a mixture of 25 and 26. Treatment of 24 with TFA cleavage cocktail (95% TFA, 2.5% TIPS and 2.5% H₂O) catalyzes a cyclization and loss of water to form 26.

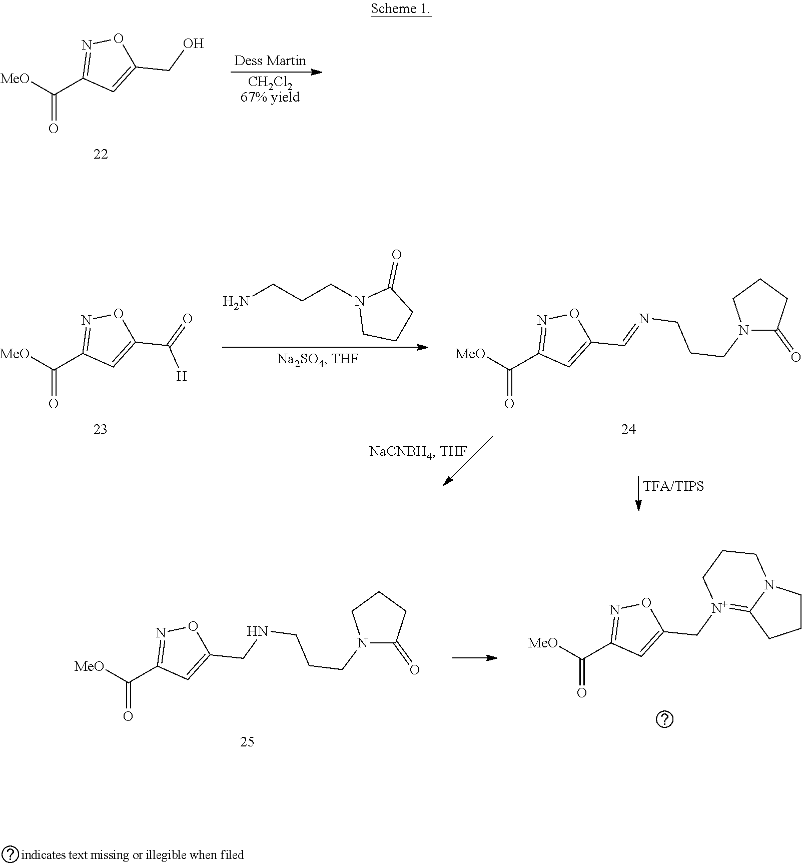

Characterization of Compound 26.

¹H NMR (400 MHz, CDCl₃); δ 7.0 (s, 1H), 4.44 (s, 2H), 3.96 (s, 3H). 3.47-3.38 (m, 4H), 3.05 (t, J=6.6 Hz, 2H), 2.42 (t, J=8.04 Hz, 2H), 2.12-2.10 (m, 4H)

¹³C NMR (400 MHz, CDCl₃); δ 177.18, 164.66, 159.64, 156.86, 107.08, 53.21, 48.20, 44.97, 41.64, 39.62, 30.67, 24.14, 17.93

Affinity Purification and Western Analysis of Active TB Patient Antibodies.

2-B was covalently linked to an agarose SulfoLink affinity column (ThermoFisher, Scientific) according to the manufacturer's protocol. Briefly, resin slurry (2 mL) was added to a fritted syringe (5 mL) and evacuated by centrifugation. The resin was washed (50 mM Tris, 5 mM EDTA, pH 8.5, 3×2 mL). 2-B was dissolved (2 μM in PBS) added to the column, the column was incubated and with rotation (1 h, RT, 8 rpm), and washed (1 M NaCl, PBS, 3×2 mL). Cysteine solution (50 mM cysteine, 50 mM Tris, 5 mM EDTA, pH 8.5, 2 mL) was added and the column was incubated with rotation (15 min, RT, 8 RPM) The column was thoroughly flushed and equilibrated into TBS. ATB patient serum (50 μL) was diluted (1:10 in TBS), the diluted sample was added to the affinity column, and the column incubated with rorpmtation (1 h, RT, 8 rpm). The column was washed (TBS, 3×2 mL), IgG elution buffer (0.2 M glycine-HCl, pH 2.5-3.0, 0.5 mL) was added, incubated briefly with the column (1 min, RT), removed, and immediately neutralized (1 M Tris pH 9, 50 μL). Sample was exchanged to TBS via size exclusion according to manufacturer protocols (PD-10, GE Life Sciences. Pittsburgh, Pa.), concentrated (˜100 μg/mL total protein), and BSA (0.1%) was added to yield purified ATB patient antibody solution.

Laemmli sample buffer was added to each of the following: native Ag85B (1 μg), Mtb H37Rv culture filtrate proteins (10 μg), and Mtb strain CDC1551 (10 μg, BEI Resources). The samples were heated (5 min, 95° C.). Samples were analyzed by SDS-PAGE (4-20% Mini-PROTEAN TGX, Bio-Rad, 200 V, 45 min), and immunoblotted onto a nitrocellulose membrane (Trans-Blot Turbo Transfer System, Bio-Rad Laboratories, Inc Hercules. Calif.). The membrane was washed (0.1 M Tris, 0.2% Tween-20, pH 7.5, 1 h, 4° C.), then incubated in a fresh aliquot of the same buffer (overnight, 4° C.). The membrane was washed (0.1 M Tris, 0.2% Tween-20. pH 7.5, 4×24 h each). The membrane was blocked (1% BSA, 0.2% Tween-20, 1 h, RT). The purified ATB patient antibody solution (250 μL) and blocking solution (1% BSA, 0.2% Tween-20) were added to the membrane and the membrane was incubated (overnight, 4° C.). The membrane was washed (TBST, 4×5 min), goat anti-human IgG HRP conjugate (1:10,000 dilution in TBST, 1% BSA, ThermoFisher) was added to the membrane and the membrane was incubated (1 h, RT). The membrane was washed (TBST, 4×5 min), HRP substrate was added (SuperSignal West Pico Chemiluminescent substrate, ThermoFisher), and the membrane was visualized (Typhoon 9410 Variable Mode Imager, GE Healthcare Life Sciences, Pittsburgh, Pa.).

Another blot was performed as described above and probed with anti-Ag85 (Polyclonal Anti-Mycobacterium tuberculosis Antigen 85 Complex, 1:1000 dilution in 1% BSA, 0.2% Tween-20, BEI Resources, Manassas, Va.).

Native Ag85B-Based ELISA.

Ag85B (10 μg/mL, PBS, BE Resources) was incubated in ELISA plates (Greiner Lumitrac 600 flat bottom white polystyrene, 100 μL, overnight, 4° C.). Wells were washed (PBST, 3×150 μL), and blocked with PBS StartingBlock (100 μL, 1 h, RT). Patient serum samples were diluted (800 μg/ml in PBS StartingBlock), added to the plate (100 μL), and incubated (4 h, RT). Wells were washed (PBST, 3×150 μL). Goat anti-human IgG-HRP was added (100 μL, 1:40,000 in PBS StartingBlock, Life Technologies), the plate was incubated (1 h, RT), and wells were washed (PBST, 3×150 μL). ELISA Supersignal Pico Chemiluminescent Substrate (ThermoFisher) was used per manufacturer's instructions and signal was quantified (Tecan Infinite M1000 Pro, Tecan Systems, Inc., San Jose, Calif.).

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps, some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a,” “an,” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A polynucleotide encoded chemical library comprising a plurality of bead members, wherein each bead member comprises:
a. a chemical moiety comprising a compound library member;
b. a polynucleotide moiety comprising an oligonucleotide encoding the compound library member, and a barcode identifying the bead; and
c. a linking moiety linking the chemical moiety to the polynucleotide moiety.

2. The polynucleotide encoded chemical library of claim 1, wherein the barcode identifying the bead is an oligonucleotide.

3. The polynucleotide encoded chemical library of claim 1, wherein the polynucleotide is a DNA oligonucleotide.

4. The polynucleotide encoded chemical library of claim 1, comprising two or more bead members having the identical compound library member structure, identical oligonucleotide encoding the compound library member, but different barcodes identifying each bead.

5. The polynucleotide encoded chemical library of claim 4, wherein presence of identical compound library members in more than one bead while having different barcodes identifying each bead enables discriminating between the two or more beads carrying the same compound library member structure.

6. The polynucleotide encoded chemical library of claim 1, wherein the barcode identifying the bead comprises an oligonucleotide having a length of 2 to 20 nucleotides.

7. The polynucleotide encoded chemical library of claim 1, wherein barcode identifying the bead comprises an oligonucleotide having a length of 2 to 50 nucleotides.

8. The polynucleotide encoded chemical library of claim 1, wherein barcode identifying the bead is an oligonucleotide and is prepared by split-and-pool combinatorial ligation or by split-and-pool enzymatic ligation reaction.

9. The polynucleotide encoded chemical library of claim 1, wherein the polynucleotide moiety is synthesized in solid phase on the beads.

10. The polynucleotide encoded chemical library of claim 1, wherein the oligonucleotide encoding the compound library member is ligated in parallel with the compound library member synthesis.

11. The polynucleotide encoded chemical library of claim 8, wherein polynucleotide encoded split-and-pool synthesis proceeds with alternating steps of monomer coupling followed by oligonucleotide ligation based encoding.

12. The polynucleotide encoded chemical library of claim 1, wherein bead barcoding occurs prior to encoded library synthesis or after encoded library synthesis.

13. The polynucleotide encoded chemical library of claim 1, wherein bead barcoding occurs discontinuously, wherein portions of the barcode are installed before and after the encoded library synthesis.

14. The polynucleotide encoded chemical library of claim 1, wherein the oligonucleotide sequences encoding the compound library member and/or identifying the bead are thermodynamically optimized.

15. The polynucleotide encoded chemical library of claim 1, wherein the oligonucleotide sequences encoding the compound library member and/or identifying the bead (a) possess Hamming string distances ≥3 and/or (b) has a total read length <100 bases for facile sequencing.

16. The polynucleotide encoded chemical library of claim 1, wherein the linker moiety comprises a chromophore.

17. (canceled)

18. The polynucleotide encoded chemical library of claim 1, wherein the linker moiety comprises a chemical moiety that enhances mass spectrometric ionization efficiency.

19. (canceled)

20. The polynucleotide encoded chemical library of claim 1, wherein the linker moiety comprises an alkyne for copper catalyzed azide-alkyne cycloaddition click chemistry.

21. A method of combinatorial screening comprising the steps of:
a. Incubating a labeled protein with a polynucleotide encoded chemical library comprising a plurality of bead members, wherein the beads comprise:
i. a chemical moiety comprising a compound library member;
ii. a polynucleotide moiety comprising: an oligonucleotide encoding the compound library member structure and/or chemical synthesis history, and a barcode identifying the bead; and
iii. a linking moiety, linking the chemical moiety to the polynucleotide moiety;
b. washing the beads to remove excess unbound protein;
c. sorting and detecting the beads that have bound to the labeled protein;
d. amplifying the polynucleotide encoding sequences of the hit beads using PCR;
e. sequencing the polynucleotide moiety; and
f. identifying the hit compound library member structure based on the sequence of the oligonucleotide encoding the compound library member structure and/or synthesis history.

22-24. (canceled)

25. A method of yielding a diagnostic panel of molecules for a disease comprising:
g. providing a sample from a patient afflicted with the disease, and sample from a control individual not afflicted with the disease;
h. screening the samples against the polynucleotide encoded chemical library of claim 1;
i. utilizing a tag to label hit compound beads for fluorescence-activated cell sorting (FACS);
j. deep sequencing all hits to determine the structure of the hit compounds and each hit's occurrence frequency;
k. pruning patient hits from the control hits; and
l. resynthesizing the patient hits to yield a diagnostic panel for the disease.

26-34. (canceled)
